(19) United States
(12) Patent Application Publication
(10) Pub. No.: US 2023/0363704 A1
(43) Pub. Date: Nov. 16, 2023

(54) APPARATUSES AND METHODS FOR INTRAORAL APPLIANCES WITH SENSING DEVICES

(71) Applicant: Align Technology, Inc., San Jose, CA (US)

(72) Inventors: Zijie ZHU, Santa Clara, CA (US); Wesly WONG, Cupertino, CA (US); Byong-Ho PARK, San Jose, CA (US); Fred TING, San Jose, CA (US); Yaser SHANJANI, Milpitas, CA (US); Zhichao ZHANG, Walnut Creek, CA (US); Kangning SU, San Jose, CA (US); Hongling CHEN, Santa Clara, CA (US); Jun SATO, San Jose, CA (US); Srinivas KAZA, Mountain View, CA (US); Maria LVOVA, Yerevan (AM); Olga LIKHACHEVA, Moscow (RU)

(21) Appl. No.: 18/316,228

(22) Filed: May 11, 2023

Related U.S. Application Data
(60) Provisional application No. 63/340,925, filed on May 11, 2022.

(51) Int. Cl. A61B 5/00; A61C 7/00; A61C 7/08

(52) U.S. Cl. CPC A61B 5/4833; A61C 7/002; A61C 7/08

(57) ABSTRACT

Methods and apparatuses are disclosed for electronic devices associated with monitoring a patient's use of an intraoral dental appliance. The monitoring electronics may communicate with near-field communication (NFC) signals. An NFC booster is disclosed to improve NFC data transfer between the electronic devices that monitor patient usage and communication devices. NFC boosters may be included within dental appliance cases and/or mobile phone cases. Also described are method and apparatuses for mounting the monitoring electronics onto the dental appliance. Also described are compact sensors, e.g., capacitive sensors, that may be included as part of a monitoring electronics unit.

### CLAIM OF PRIORITY

This patent application claims priority to U.S. provisional patent application 63/340,925, titled “APPARATUS AND METHODS FOR INTRAORAL APPLIANCES WITH SENSING DEVICES,” and filed on May 11, 2022, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Orthodontic procedures typically involve repositioning a patient's teeth to a desired arrangement in order to correct malocclusions and/or improve aesthetics. To achieve these objectives, dental appliances such as braces, shell aligners, and the like can be applied to the patient's teeth by an orthodontic practitioner. The appliance can be configured to exert force on one or more teeth in order to effect desired tooth movements according to a treatment plan.

During orthodontic treatment with patient-removable appliances, the practitioner may rely on the patient to comply with the prescribed appliance usage. In some instances, a patient may not wear the dental appliance as prescribed by the practitioner. Extended removal of the appliance, for any reason beyond what is recommended, may interrupt the treatment plan and lengthen the overall period of treatment. There is a need for methods and apparatuses that allow monitoring of the wearing and/or effects of intraoral appliances.

Described herein are methods and apparatuses for performing such monitoring as well as collecting the monitoring information.

### SUMMARY OF THE DISCLOSURE

This disclosure relates to dental appliances, and more particularly to apparatuses and methods for transferring data to and from one or more monitoring electronics units associated with the dental appliances.

Described herein are various apparatuses (e.g., systems, devices, methods, or the like) that can enable or improve data transfer between a monitoring device and a communication device. A monitoring device may be mounted on a dental appliance, such as a dental aligner. The monitoring device may determine and track the time that a patient has worn the dental appliance.

Mounting the monitoring device onto the dental appliance may involve applying surface treatments to the dental appliance and/or a housing of the monitoring device that enhance a bond formed with laser-based welding.

For example, described herein are methods for attaching a monitoring electronics unit to a dental appliance, the method comprising: fabricating the dental appliance, wherein the dental appliance includes a mounting surface at a mounting location determined by a mounting location optimization protocol; positioning a housing at least partially enclosing the monitoring electronics unit housing on the mounting surface; and welding the housing to the mounting surface.

Any of these methods may include determining the mounting surface at the mounting location by a mounting location optimization protocol comprising: optimizing a location of the mounting surface in a digital model of the dental appliance by: iteratively adjusting one or more of a position, an angulation and an orientation of the mounting surface relative to a patient's dental arch corresponding to the digital model of the patient's dentition, until each of the position, the angulation and the orientation of the mounting surface in the digital model of the dental appliance are within a constrained range relative to the digital model of the patient's dentition and the mounting surface does not collide with the patient's teeth or with a treatment feature of the dental appliance. Any of these methods may include placing the monitoring electronics unit into a housing. Any of these methods may include preparing mounting surfaces of the housing. The dental appliance may comprise an aligner. The monitoring electronics unit may comprise an electronics compliance indicator (ECI).

In any of these methods, preparing the mounting surfaces may include applying an infrared-absorbent spray to the mounting surfaces of the housing. Any of these methods may include applying an infrared-absorbent spray to mounting surfaces of the aligner. Positioning the housing may include applying clamping force to the housing. Welding may include heating the housing and the aligner via a laser (e.g., a Yttrium-Aluminum Garnet laser).

Also described herein are methods for attaching a monitoring electronics unit to a dental appliance, the method comprising: fabricating the dental appliance, wherein the dental appliance includes a mounting surface at a mounting location determined by a mounting location optimization protocol, wherein the mounting location optimization protocol: optimizing a location of the mounting surface in a digital model of the dental appliance by iteratively adjusting one or more of a position, an angulation and an orientation of the mounting surface relative to a patient's dental arch corresponding to the digital model of the patient's dentition, until each of the position, the angulation and the orientation of the mounting surface in the digital model of the dental appliance are within a constrained range relative to the digital model of the patient's dentition and the mounting surface does not collide with the patient's teeth or with a treatment feature of the aligner; placing the monitoring electronics unit into a housing; preparing mounting surfaces of the housing; positioning a housing at least partially enclosing the monitoring electronics unit housing on the mounting surface; and welding the housing to the mounting surface.

Also described herein are methods of setting a location of a mounting surface for a monitoring electronics unit on a dental appliance, the method comprising: optimizing the location of the mounting surface having a predetermined mounting area in a digital model of the dental appliance by: iteratively adjusting one or more of a position, an angulation and an orientation of the mounting surface relative to a patient's dental arch corresponding to a digital model of the patient's dentition corresponding to the digital model of the dental appliance, until each of the position, the angulation and the orientation of the mounting surface in the digital model of the dental appliance are each within a constraint range relative to the digital model of the patient's dentition, and the mounting surface does not collide with the patient's teeth or with a treatment feature of the aligner; and outputting the digital model of the dental appliance, or a digital model of a mold for forming the dental appliance, including the optimized location of the mounting surface. Any of these methods may include iteratively adjusting the position by iteratively adjusting a distance from the mounting surface to a tooth buccal surface, further wherein the constraint range of the distance from the mounting surface to the tooth buccal surface is between 0.2 and 1.0 mm. Iteratively adjusting the position may include iteratively adjusting a distance from the mounting surface to a gingiva surface, further wherein the constraint range of the distance from the mounting surface to the gingiva surface is between 0.5 and 1.0 mm. Iteratively adjusting the position may include iteratively adjusting a distance from an occlusal edge of the mounting surface to a buccal cusp, further wherein the constraint range of the distance from the occlusal edge of the mounting surface to the buccal cusp is less than 1.0 mm. Iteratively adjusting the position may include iteratively adjusting a distance from an edge of the mounting surface to a treatment feature of the aligner, further wherein the constraint range of the distance from the occlusal edge of the mounting surface to the buccal cusp is 0.2 mm or more. Iteratively adjusting the position may include iteratively adjusting until the mounting surface does not collide with teeth on opposite jaw or with neighboring teeth. Iteratively adjusting the orientation may comprise iteratively adjusting an orientation between a normal of the mounting surface and a normal to a jaw arch of the patient's dentition, further wherein the constraint range of the orientation between the normal of the mounting surface and the normal to a jaw arch of the patient's dentition is between −12.5 and 12.5 degrees. Iteratively adjusting the angulation may comprise iteratively adjusting an orientation between a normal of the occlusal edge of the mounting surface and a normal to a jaw occlusal plane of the patient's dentition, further wherein the constraint range of the orientation between the normal of the occlusal edge of the mounting surface and the normal to the jaw occlusal plane is between −12.5 and 12.5 degrees.

Any of these methods may include adding a gingival buffer region around at least a portion of the mounting surface in the digital model of the dental appliance including the optimized location of the mounting surface. Any of these methods may include manufacturing the dental appliance from the digital model of the dental appliance including the optimized location of the mounting surface. Any of these methods may include repeating the optimizing and outputting steps for each of plurality of dental appliances in a sequence of dental appliances of a treatment plan. The change in position of the mounting surface between subsequent of the dental appliances in the series of dental appliances may be constrained in order to smooth the transition of the mounting surface over the series of dental appliances.

Also described herein are methods of setting a location of a mounting surface for a monitoring electronics unit on a dental appliance, the method comprising: receiving a digital model the dental appliance and a digital model of the patient's dentition; optimizing the location of the mounting surface having a predetermined mounting area in the digital model of the dental appliance by: starting from an initial location of the mounting surface, iteratively adjusting one or more of a position, an angulation and an orientation of the mounting surface relative to a patient's dental arch corresponding to the digital model of the patient's dentition, until each of the position, the angulation and the orientation of the mounting surface in the digital model of the dental appliance are within a constrained range relative to the digital model of the patient's dentition, and the mounting surface does not collide with the patient's teeth or with a treatment feature of the aligner; outputting the digital model of the dental appliance, or a digital model of a mold for forming the dental appliance, including the optimized location of the mounting surface; and manufacturing the dental appliance from the digital model of the dental appliance including the optimized location of the mounting surface.

Also described herein are dental appliances made by any of these methods. For example, described herein are dental appliance devices, including: a body comprising a tooth receiving cavity configured to receive teeth of a patient's dental arch, the body having a buccal side, a lingual side and an occlusal side; a mounting surface comprising a flat outer surface, further wherein the mounting surface is positioned on the buccal side of the body so that the flat outer surface has an angulation and an orientation that is within a constraint range relative to the patient's dentition when the dental appliance is worn by the patient; and a monitoring electronics unit mounted onto the mounting surface, further wherein the mounting surface is configured so that a monitoring electronics unit mounted to the mounting surface does not collide with the patient's teeth when worn. For example, in any of these devices distance from the mounting surface to a tooth buccal surface when the dental appliance is worn is between 0.2 and 1.0 mm. A distance from the mounting surface to a gingival surface of the patient may be between 0.5 and 1.0 mm when the device is worn. A distance from an occlusal edge of the mounting surface to a buccal cusp of the body may be less than 1.0 mm. A distance from an edge of the mounting surface to a treatment feature of the aligner may be 0.2 mm or more. A normal to a jaw arch of the patient's dentition when the dental appliance is worn may deviate from a normal of the mounting surface by between −12.5 and 12.5 degrees. A difference between a normal of the occlusal edge of the mounting surface and a normal to a jaw occlusal plane through the body may be between −12.5 and 12.5 degrees.

These monitoring devices may use near-field communication (NFC) signals and protocols to transfer data, including dental appliance monitoring data, to the communication device. NFC data transfer may be difficult due in part the size of the antenna associated with the monitoring device.

NFC boosting devices (referred to as NFC boosters) may improve communications between monitoring devices and communication devices. The NFC boosters may include antennas and impedance matching circuits to provide enhanced NFC data transfer. Some NFC boosters may be passive (e.g., require no power source). In some examples, the NFC boosters may be integrated into phone cases or aligner cases.

Any of the systems described herein may implement an NFC booster system. The NFC booster system may include at least two antennas and a matching circuit that is configured to match an impedance of a first antenna to an impedance of a second antenna.

For example, described herein are near-field communication (NFC) booster system comprising: a base configured to receive a dental appliance in an inner chamber therein, wherein the dental appliance includes a monitoring electronics unit configured to communicate via near-field communication (NFC) signals; a lid coupled to the base and configured to open to provide access to the inner chamber in the base, wherein the lid is configured to seat a smartphone thereon when closed; a first antenna configured to have a first impedance and configured to communicate with NFC signals, wherein the first antenna includes a multi-turn coil with a diameter of less than one centimeter (cm) that is configured to communicate with the monitoring electronics unit within the base; a second antenna in the lid and configured to have a second impedance that is different from the first impedance and that is configured to communicate with NFC signals, wherein the second antenna is configured to communicate with a mobile phone, a tablet computer, a laptop computer, or a combination thereof; and one or more matching circuits coupled to the first antenna and configured to match the impedance of the first antenna to the impedance of an antenna of the monitoring electronics unit and coupled to the second antenna and configured to match the impedance of the second antenna to the impedance of an antenna of the mobile phone, tablet computer, laptop computer, or combination thereof.

Any of the systems described herein may include a first antenna configured to have a first impedance and communicate with NFC signals, a second antenna configured to have a second impedance different from the first impedance and communicate with NFC signals, and a matching circuit configured to couple the first antenna to the second antenna and match the first impedance to the second impedance.

In any of the systems described herein, the first antenna may be configured to communicate with a monitoring electronics unit. For example, the monitoring electronics unit may be an electronic compliance indicator (ECI). In some examples, any of the systems described herein an ECI may monitor and log (e.g., record) a patient's use of a device, including intraoral devices such as dental aligners. Furthermore, in some examples, an ECI may be configured to monitor and log patient use of an intraoral device based at least in part on a capacitance sensed by the ECI. Although the disclosure focuses on ECIs as an example, the disclosure contemplates that any suitable monitoring electronics unit may be employed, not limited to ECIs, including individual sensors or groups of sensors. Example monitoring electronics units may include a temperature sensor, a pH sensor, a glucose sensor, a pressure sensor, a capacitance sensor, or any other suitable sensor, and may be used for any suitable purpose, including monitoring and/or providing recommendations and/or notifications on patient health.

In any of the systems described herein, the first antenna may include a multi-turn coil with a diameter of approximately one centimeter. In some examples, the first antenna may include two or more multi-turn coil antennas. Furthermore, in any of the systems described herein, the two or more multi-turn coil antennas may be coupled simultaneously to the matching circuit.

In any of the systems described herein, the first antenna may include a plurality of antennas and one of the plurality of antennas may be selectively coupled to the matching circuit. In some examples, the second antenna may be configured to communicate with a mobile phone, a tablet computer, a laptop computer, or a combination thereof. In any of the systems described herein, the second antenna may include a multi-turn coil with a diameter of approximately five centimeters.

In any of the systems described herein, the matching circuit may include a battery, a power management unit, and a voltage regulator configured to provide power to the matching circuit. Furthermore, in some examples, the first antenna and the second antenna may be configured to transmit and receive radio signals having a frequency of approximately 13.56 MHz.

In any of the systems described herein, the first antenna, the second antenna, and the matching circuit may be integrated into a clip configured to contact a screen and a back of the communication device. In some examples, the first antenna may be disposed on a first end of the clip and the second antenna may be disposed on a second end of the clip, opposite the first end.

Any of the systems described herein may further include a mobile phone case, where the first antenna, the second antenna, and the matching circuit of an NFC booster system are disposed within the mobile phone case. In some examples, the first antenna may be disposed on a flap of the mobile phone case and the second antenna may be disposed in a region configured to receive a communication device.

In any of the systems described herein, the system may further include a pad including a first section and a second section, where the first section includes the first antenna and the second section includes the second antenna. In some examples, the second section may include a non-contact charging coil for a communication device.

Any of the apparatuses described herein may include a case configured to contain and/or store a dental aligner. The case may include a base configured to receive a dental aligner, where the dental aligner includes a monitoring electronics unit (e.g., an ECI) configured to communicate via near-field communication (NFC) signals, a lid movably coupled to the base and configured to pivot open and provide access to an inner chamber in the base, and an NFC booster. The NFC booster may include a first antenna having a first impedance and configured to communicate via NFC signals, a second antenna having a second impedance different from the first impedance configured to communicate with NFC signals, and a matching circuit configured to couple the first antenna to the second antenna and match the first impedance to the second impedance.

In any of the apparatuses described herein, the first antenna may be affixed to the lid and the second antenna may affixed to the base. In some examples, the second antenna may be affixed to an inner circumference of the inner chamber in the base. In some further examples, the second antenna may include a single multi-turn loop of conductive material. In some examples, the second antenna may include a plurality of separate multi-turn loops of conductive material. In any of the apparatuses described herein, the second antenna may be affixed to a bottom surface of the inner chamber in the base. In some examples, the second antenna may include a single multi-turn loop of conductive material. In some other examples, the second antenna may include a plurality of separate multi-turn loops of conductive material.

In any of the apparatuses described herein, the second antenna may include two or more circular-shaped multi-turn coils that are configured to be concentric with each other, two or more oval-shaped multi-turn coils that are configured to be concentric with each other, or a combination thereof. Furthermore, the second antenna may include two or more antennas that are coupled simultaneously to the matching circuit.

In any of the apparatuses described herein, the lid may include a conductive shield disposed on an inner surface. Also, in any of the apparatuses described herein, the base may include an aligner guide disposed within the inner chamber in the base and configured to position an NFC device within the case. Any of the aligner guides may be replaceable by a user. Furthermore, any of the aligner guides may be configured to conform to any aligners. In any of the aligner guides, they may be configured to position the NFC device adjacent to the second antenna.

Any of the apparatuses described herein, may include a matching circuit that further includes a battery, a power management unit, and a voltage regulator configured to provide power to the matching circuit. Furthermore, in any of the apparatuses described herein, the lid may be configured to receive a communication device.

Any of the methods described herein may include positioning a NFC device proximate to a first antenna of an NFC booster, positioning a communication device proximate to a second antenna of the NFC booster, receiving by the first antenna, NFC signals from the NFC device, and transmitting, by the second antenna, NFC signals received from the NFC device to the communication device.

Any of the methods described herein may further include matching, by the NFC booster an impedance of the first antenna and an impedance of the second antenna. Furthermore, the communication device may include a tablet computer, laptop computer, mobile phone, or a combination thereof.

In any of the methods described herein, the NFC device may include a monitoring electronics unit. For example, the NFC device may include an ECI configured to monitor and log patient use of an intraoral device based at least in part on a capacitance, a pressure, temperature, and/or any other suitable parameter sensed by the ECI. In any of the methods, the first antenna may include a multi-turn coil with a diameter of approximately one centimeter. The first antenna may further include two or more multi-turn coil antennas and the two or more multi-turn coil antennas are coupled simultaneously to the NFC booster. In some examples, the first antenna may include a plurality of antennas and wherein one of the plurality of antennas is selectively coupled to the NFC booster. In some examples, the NFC booster may include a battery, a power management unit, and a voltage regulator configured to provide power to the NFC booster.

Any of the methods described herein may include loading a housing with an electronic monitoring device, preparing mounting surfaces of the housing, positioning the housing on the aligner, and welding the housing to the aligner.

In any of the methods described herein, the electronic monitoring device may be an electronic compliance indicator (ECI) to determine and log use of the aligner by a patient based at least in part on a sensed capacitance or temperature.

In any of the methods described herein, preparing the mounting surfaces may include applying an infrared-absorbent spray to the mounting surfaces of the housing. Furthermore, the infrared-absorbent spray may be applied to mounting surfaces of the aligner.

In any of the methods described herein, positioning the housing may include applying a clamping force to the housing. Furthermore, in any of the methods, the welding may include heating the housing and the aligner via a laser. The laser may be a Yttrium-Aluminum Garnet laser.

Any of the sensors described herein may include a battery, a first electrode disposed on a printed circuit board (PCB), a second electrode, and electronic components powered by the battery and configured to determine at least one of a capacitance or a temperature sensed by the first electrode and the second electrode. Any of the sensors may include electronic components configured to monitor and log patient use of an intraoral device based at least in part on the sensed capacitance and/or sensed temperature.

In any of the sensors described herein, the second electrode may be an electrode coated onto a plastic enclosure. In any of the sensors described herein, one terminal of the battery may be configured to operate as the second electrode. The one terminal of the battery may be the positive terminal.

Any of the sensors described herein may include a third electrode disposed on the printed circuit board and coupled in parallel with the second electrode. Furthermore, the first and third electrodes may be on one side of the PCB. In some examples, first and third electrodes may be interdigitated. In any of the sensors described herein, the second electrode may have a surface area comparable to a surface area sum of the electronic components.

For example, described herein are near-field communication (NFC) booster systems comprising: a housing; a first antenna configured to have a first impedance and configured to communicate with NFC signals, wherein the first antenna includes a multi-turn coil with a diameter of less than one centimeter (cm) that is configured to communicate with the monitoring electronics unit; a second antenna configured to have a second impedance that is different from the first impedance and that is configured to communicate with NFC signals, wherein the second antenna is configured to communicate with a mobile phone, a tablet computer, a laptop computer, or a combination thereof; and one or more matching circuits coupled to the first antenna and configured to match the impedance of the first antenna to the impedance of an antenna of the monitoring electronics unit and coupled to the second antenna and configured to match the impedance of the second antenna to the impedance of an antenna of the mobile phone, tablet computer, laptop computer, or combination thereof.

The monitoring electronics unit may be configured to monitor and log patient use of an intraoral device. The first antenna may include two or more multi-turn coil antennas. The two or more multi-turn coil antennas may be coupled simultaneously to the matching circuit. The first antenna may include a plurality of antennas and wherein one of the plurality of antennas is selectively coupled to the matching circuit. The second antenna may include a multi-turn coil with a diameter of 5 cm or greater. The one or more matching circuits may include a battery, a power management unit, and a voltage regulator configured to provide power to the one or more matching circuits. The first antenna and the second antenna may be configured to transmit and receive radio signals with a frequency of approximately 13.56 MHz. The housing may enclose the first antenna, the second antenna, and the matching circuit. In some examples the housing is configured as a clip. The housing may be configured as a smartphone case or a pad. In some examples the second antenna is disposed in a region configured to receive a communication device.

For example, described herein is a case comprising: a base configured to receive a dental appliance in an inner chamber therein, wherein the dental appliance includes a monitoring electronics unit configured to communicate via near-field communication (NFC) signals; a lid coupled to the base and configured to open to provide access to the inner chamber in the base, wherein the lid is configured to seat a smartphone thereon when closed; and an NFC booster comprising: a first antenna within the lid, the first antenna having a first impedance; and a matching circuit coupled to the first antenna, wherein the first antenna and matching circuit are configured to receive data via NFC signals from the monitoring electronics unit held within the inner chamber and to relay the data by NFC to a smartphone seated on the lid. Advantageously, the methods an apparatuses described herein may include boosters (e.g., NFC boosters) that may be passive, meaning that they may not use or need a power source, such as battery, but may be passively powered by the application of the electric field that may trigger and allow the very low power and surprisingly robust operation of these apparatuses.

The first antenna and matching circuit may be passive circuits. In general, the case may include a power source, wherein the matching circuit and first antenna are actively powered by the power source. The matching circuit may include a power management unit, and a voltage regulator configured to provide power to the matching circuit. The first antenna may include a single multi-turn loop of conductive material. In some examples the first antenna includes a plurality of separate multi-turn loops of conductive material. The first antenna may include two or more circular-shaped multi-turn coils that are configured to be concentric with each other, two or more oval-shaped multi-turn coils that are configured to be concentric with each other, or a combination thereof. The first antenna may include two or more antennas that are coupled simultaneously to the matching circuit.

The lid may include a conductive shield disposed on an inner surface. The base may include a guide disposed within the inner chamber in the base and configured to position an NFC device within the case adjacent to the first antenna. The guide may be configured to be replaceable by a user. The guide may be configured to conform to the dental appliance.

Also described herein are methods of operating these apparatuses. For example, a method for coupling a dental appliance to a handheld processing device by near-field communication (NFC) communications may include: positioning the dental appliance including a monitoring electronics unit having a first NFC antenna adjacent to a second NFC antenna of an NFC booster device, wherein the second NFC antenna includes a multi-turn coil with a diameter of less than one centimeter (cm); positioning the handheld processing device so that a fourth NFC antenna of the handheld processing device is adjacent to a third NFC antenna of the booster device; and receiving, using a first matching circuit coupled to the second NFC antenna, data from the monitoring electronics unit; and transmitting the data, using a second matching circuit coupled to the third NFC antenna and to the first matching circuit, to the handheld processing device.

The handheld processing device may comprise one or more of: a mobile phone, a tablet computer, a laptop computer, a wearable device (e.g., a smart watch/band), AR/VR devices (e.g., AR glasses), or a combination thereof. The second NFC antenna, the third NFC antenna and the first and second matching circuits may be passive NFC circuits. The monitoring electronics unit may be configured to monitor and log patient use of an intraoral device based at least in part on a capacitance sensed by the monitoring electronics unit. The monitoring electronics unit may include an electronic compliance indicator (ECI) configured to monitor and log patient use of an intraoral device based at least in part on a temperature sensed by the ECI. The first antenna may include a multi-turn coil with a diameter of one centimeter (cm) or less. The second antenna may include two or more multi-turn coil antennas and the two or more multi-turn coil antennas are coupled simultaneously to the NFC booster device. In general, these device may be passive, as mentioned above. For example, the NFC booster device may not include a power supply. Alternatively, the NFC booster device may include a battery, a power management unit, and a voltage regulator configured to provide power to the NFC booster. Positioning the dental appliance may include positioning the dental appliance within a case so that the monitoring electronics unit is held in a predefined location.

Also described herein are sensors, and particularly compact capacitive sensors for an intraoral device (or for any appropriate use). These sensors may include: a first electrode disposed on a printed circuit board (PCB); a battery having a first surface configured as an anode or cathode, wherein the battery is coupled to the PCB; a second electrode comprising the first surface of the battery; and electronic components powered by the battery and configured to determine at least one of a capacitance or a temperature sensed by the first electrode and the second electrode.

The electronic components may be configured to monitor and log patient use of an intraoral device based at least in part on the sensed capacitance. The electronic components may be configured to monitor and log patient use of an intraoral device based at least in part on the sensed temperature.

The second electrode may be an electrode coated onto a plastic enclosure. The terminal of the battery may be a positive battery terminal. Any of these device may include a third electrode disposed on the printed circuit board and coupled in parallel with the second electrode. The first and third electrodes may be on one side of the PCB. The first and third electrodes may be interdigitated. The second electrode may have a surface area comparable to the sum of the surface area of the electronic components.

Also described herein are methods for attaching a monitoring electronics unit to a dental appliance, the method comprising: fabricating the dental appliance, wherein the dental appliance includes a mounting surface at a mounting location determined by a mounting location optimization protocol; positioning a housing at least partially enclosing the monitoring electronics unit housing on the mounting surface; and welding the housing to the mounting surface. Any of these methods may include determining the mounting surface at the mounting location by a mounting location optimization protocol comprising: optimizing a location of the mounting surface in a digital model of the dental appliance by: iteratively adjusting one or more of a position, an angulation and an orientation (including rotation) of the mounting surface relative to a patient's dental arch corresponding to the digital model of the patient's dentition, until each of the position, the angulation and the orientation of the mounting surface in the digital model of the dental appliance are within a constrained range relative to the digital model of the patient's dentition and the mounting surface does not collide with the patient's teeth or with a treatment feature of the dental appliance. The method may include placing the monitoring electronics unit into a housing. Any of these methods may include preparing mounting surfaces of the housing. For example, preparing the mounting surfaces may include applying an infrared-absorbent spray to the mounting surfaces of the housing. Any of these methods may include applying an infrared-absorbent spray to mounting surfaces of the aligner. Positioning the housing may include applying clamping force to the housing. Welding may include heating the housing and the aligner via a laser (e.g., a Yttrium-Aluminum Garnet laser, etc.).

For example, a method for attaching a monitoring electronics unit to a dental appliance may include: fabricating the dental appliance, wherein the dental appliance includes a mounting surface at a mounting location determined by a mounting location optimization protocol, wherein the mounting location optimization protocol: optimizing a location of the mounting surface in a digital model of the dental appliance by iteratively adjusting one or more of a position, an angulation and an orientation (including rotation) of the mounting surface relative to a patient's dental arch corresponding to the digital model of the patient's dentition, until each of the position, the angulation and the orientation of the mounting surface in the digital model of the dental appliance are within a constrained range relative to the digital model of the patient's dentition and the mounting surface does not collide with the patient's teeth or with a treatment feature of the aligner; placing the monitoring electronics unit into a housing; preparing mounting surfaces of the housing; positioning a housing at least partially enclosing the monitoring electronics unit housing on the mounting surface; and welding the housing to the mounting surface.

Also described herein are methods of setting a location of a mounting surface for a monitoring electronics unit on a dental appliance, the method comprising: optimizing the location of the mounting surface having a predetermined mounting area in a digital model of the dental appliance by: iteratively adjusting one or more of a position, an angulation and an orientation of the mounting surface relative to a patient's dental arch corresponding to a digital model of the patient's dentition corresponding to the digital model of the dental appliance, until each of the position, the angulation and the orientation of the mounting surface in the digital model of the dental appliance are each within a constraint range relative to the digital model of the patient's dentition, and the mounting surface does not collide with the patient's teeth or with a treatment feature of the aligner; and outputting the digital model of the dental appliance including the optimized location of the mounting surface.

Iteratively adjusting the position may include iteratively adjusting a distance from the mounting surface to a tooth buccal surface, further wherein the constraint range of the distance from the mounting surface to the tooth buccal surface is between 0.2 and 1.0 mm. Iteratively adjusting the position may comprise iteratively adjusting a distance from the mounting surface to a gingiva surface, further wherein the constraint range of the distance from the mounting surface to the gingiva surface is between 0.5 and 1.0 mm. Iteratively adjusting the position may comprise iteratively adjusting a distance from an occlusal edge of the mounting surface to a buccal cusp, further wherein the constraint range of the distance from the occlusal edge of the mounting surface to the buccal cusp is up to 1.0 mm. Iteratively adjusting the position may comprise iteratively adjusting a distance from an edge of the mounting surface to a treatment feature of the aligner, further wherein the constraint range of the distance from the occlusal edge of the mounting surface to the buccal cusp is 0.2 mm or more. Iteratively adjusting the position may comprise iteratively adjusting until the mounting surface does not collide with teeth on opposite jaw or with neighboring teeth. Iteratively adjusting the orientation may comprise iteratively adjusting the orientation between a normal of the mounting surface and a normal to a jaw arch of the patient's dentition, further wherein the constraint range of the orientation between the normal of the mounting surface and the normal to a jaw arch of the patient's dentition is between −12.5 and 12.5 degrees. Iteratively adjusting the angulation may comprise iteratively adjusting the orientation between a normal of the occlusal edge of the mounting surface and a normal to a jaw occlusal plane of the patient's dentition, further wherein the constraint range of the orientation between the normal of the occlusal edge of the mounting surface and the normal to the jaw occlusal plane is between −12.5 and 12.5 degrees.

Any of these methods may include adding a gingival buffer region around at least a portion of the mounting surface in the digital model of the dental appliance including the optimized location of the mounting surface.

Any of these methods may include adjusting a trimming line to accommodate the mounting surface in the digital model of the dental appliance including the optimized location of the mounting surface, and/or manufacturing the dental appliance from the digital model of the dental appliance including the optimized location of the mounting surface.

In general, any of these methods may include repeating the step of iteratively adjusting until a fail condition is met or until each of the position, the angulation and the orientation of the mounting surface in the digital model of the dental appliance are within a constrained range relative to the digital model of the patient's dentition, and the mounting surface does not collide with the patient's teeth or with a treatment feature of the aligner. Any of these methods may include repeating the optimizing and outputting steps for each of plurality of dental appliances in a sequence of dental appliances of a treatment plan. The change in position of the mounting surface between subsequent of the dental appliances in the series of dental appliances may be constrained in order to smooth the transition of the mounting surface over the series of dental appliances.

For example, described herein are methods of setting a location of a mounting surface for a monitoring electronics unit on a dental appliance, the method comprising: receiving a digital model the dental appliance and a digital model of the patient's dentition; optimizing the location of the mounting surface having a predetermined mounting area in the digital model of the dental appliance by: starting from an initial location of the mounting surface, iteratively adjusting one or more of a position, an angulation and an orientation of the mounting surface relative to a patient's dental arch corresponding to the digital model of the patient's dentition, until each of the position, the angulation and the orientation of the mounting surface in the digital model of the dental appliance are within a constrained range relative to the digital model of the patient's dentition, and the mounting surface does not collide with the patient's teeth or with a treatment feature of the aligner; outputting the digital model of the dental appliance including the optimized location of the mounting surface; and manufacturing the dental appliance from the digital model of the dental appliance including the optimized location of the mounting surface.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A shows an example of a dental appliance (e.g., aligner) that may be prepared to receive a monitoring electronics unit (e.g., an electronic compliance indicator, or ECI) device.

FIG. 1B shows an ECI device disposed within an ECI housing.

FIG. 1C shows the housing of FIG. 1B affixed to the aligner of FIG. 1A.

FIGS. 2A-2F illustrate example steps for attaching an ECI device to an aligner.

FIGS. 2G-2I illustrate examples of constraints of positioning a mounting surface for a monitoring electronics unit (e.g., ECI) as described herein. FIG. 2G illustrates possible distances of the mounting surface from a buccal surface (e.g., top buccal surface) of tooth (e.g., between 0.2-1 mm). FIG. 2H illustrates possible distances between the mounting surface to a gingival surface of the digital model of the patient's gingiva (e.g., between 0.5-1.0 mm). FIG. 2I illustrates a distance from the buccal edge of the mounting surface to the buccal cusp (e.g., up to 1.0 mm).

FIG. 2I shows an example of the constraints of positioning the occlusal edge of the mounting surface relative to the buccal incisal points.

FIG. 2J constraints of positioning the mounting surface relative to one or more clinical features (e.g., attachments, precision cuts, etc.).

FIGS. 2K-2M illustrates constraints for including a safety gap around the mounting surface to prevent contact with the gingiva.

FIGS. 2N-2P illustrate constraints for orientation between the normal of the buccal edge surface of the mounting surface (x axis) and normal to the jaw arch within an acceptable interval.

FIGS. 2Q-2S illustrate constraints for inclination of the a normal to the mounting surface relative to a normal to the jaw arch segment, as described herein.

FIGS. 2T-2V illustrates a region of an aligner appliance including a mounting surface and a buffer region around the mounting surface to allow for trimming (e.g., including but not limited to laser trimming).

FIG. 2W-2X illustrate examples in which a trim line (e.g., cut line) is shown for trimming the dental appliance, including trimming around the mounting surface.

FIG. 3A is a flowchart showing an example method for attaching an ECI to an aligner.

FIG. 3B illustrates one example of a flowchart showing a method of determining good locations for including a flat mounting surface on the dental appliance (e.g., aligner).

FIG. 4A shows an example of a monitoring electronics unit (shown configured as an ECI in this example).

FIG. 4B shows the second side of the printed circuit board (PCB) of the ECI of FIG. 4A.

FIG. 4C shows example connections to a first electrode and a second electrode on the second side of the PCB of FIG. 4B.

FIG. 4D shows an example of a device including a modified electrode for the ECI of FIG. 4A in which an endcap (e.g., the cathode or anode) of the battery is configured as an electrode for sensing.

FIGS. 4E and 4F show graphs illustrating temperature and capacitance measurements, respectively, for a device in which the endcap of the battery is used as an electrode for the sensor, as shown in FIG. 4D.

FIG. 5 is a block diagram of an example near-field communication (NFC) system.

FIGS. 6A-6C show example NFC boosters.

FIG. 7 shows an example NFC system.

FIGS. 8A-8D show examples of antenna designs that may be used with a case.

FIGS. 9A-9C show examples of multi-coil antenna designs.

FIG. 10 shows another example antenna assembly.

FIG. 11 shows an example NFC booster system.

FIGS. 12A-12B show an example.

FIGS. 13A-13C show an example NFC system.

FIG. 14 shows another example NFC system.

FIG. 15 shows another example NFC system.

FIG. 16 shows another example NFC system.

FIG. 17 is a flowchart showing an example method for providing NFC communications.

FIGS. 18A and 18B illustrates one example of an ECI device encapsulated on an aligner.

### DETAILED DESCRIPTION

Dental appliances, such as shell aligners (sometimes referred to as dental aligners), may be used to provide a variety of dental treatments to a patient. A dental appliance may benefit by including one or more monitoring electronics units with the patient-wearable apparatus. A monitoring electronics unit may include a housing enclosing one or more sensors, circuitry (e.g., control circuitry), power sub-system, and/or wireless communications sub-system, and may be referred to as a monitoring electronics unit that may be coupled with an orthodontics appliance. Ideally the monitoring electronics unit may be included with the dental appliance in a manner that does not interfere with the operation of the dental appliance, can be comfortably worn by the patient that the dental appliance is intended to treat, and is not visible, or is minimally visible, when the dental appliance including the monitoring electronics unit is worn by the patient.

Monitoring electronics units, such as ECIs, may in general be attached or embedded to the dental appliance and may include one or more sensors to determine whether the dental appliance is being worn. The monitoring electronics unit may be integrated with the dental appliance and may connect with an external processors for receiving, analyzing, storing and/or outputting (e.g., transmitting, presenting, etc.) data related to the patient, dental appliance and/or the relationship between the patent and the dental appliance, including wearing or use information. In some cases it may be particularly beneficial for the monitoring electronics unit to connect to a booster device, which may be configured as a case or storage housing for the dental appliance (e.g., aligner, palatal expander, retainer, etc.). In some examples, the apparatus may be configured to transmit raw (or processed) data from the monitoring electronics unit when the patient puts the dental appliance on or in the case. In some examples, the appliance (e.g., aligner) wearing time may be computed based on the data recorded and transmitted. Wearing time can be shown to the patient, for example in a user interface such as an application software, and/or the dental/orthodontic professional (e.g., doctor, technician, dental assistant, etc.) can check the progress and/or health indicators. In some examples, the user interface may provide feedback on the wearing time (e.g. indicating that the appliance should be worn for longer, or to continue to wear the appliance, etc.). In some examples, the user interface may notify or alert the patient to wear the dental appliance, e.g., indicating to the patient if they forgot to put on the dental appliance. In some examples, the user interface may notify or alert the patient if it is determined that the patient is not wearing the dental appliance for a sufficient period of time. Following such a determination, in some examples, the system disclosed herein may determine a corrective action of wearing the dental appliance for a specified extra period of time, and the user interface may communicate such corrective action to the patient and/or the dental/orthodontic professional.

In general, the monitoring electronics unit may be attached directly to the dental appliance (e.g., aligner, palatal expander, retainer, etc.), and may be fully encapsulated in order to isolate the indicator. It may be particularly advantageous to include a relatively flat mounting surface region on the dental appliance onto which the monitoring electronics unit can be secured, e.g., by a welding technique. Thus, in any of these methods and apparatuses (devices, systems, etc.) described herein the shape of the dental appliance (e.g., aligner, palatal expander, retainer, etc.) may be modified to prepare a welding surface for monitoring electronics unit (e.g., ECI). Described herein are methods and apparatuses for modifying one or more (e.g., a sequence or series of) dental appliances to form a surface for mounting a monitoring electronics unit that does not interfere with the operation of the dental appliance, is optimally comfortable for the patient, allows operation of the monitoring electronics unit, and is not visible when worn. Example methods and apparatuses for mounting monitoring electronics units (e.g., ECI devices) to dental appliances (e.g., aligners) are described herein.

As used herein a dental appliance may include an aligner (e.g., a shell aligner) for repositioning a patient's teeth, a palatal expander, a retainer, etc. As used herein monitoring electronics unit may include any electronics configured and/or adapted for use with the dental appliance for monitoring one or more parameters, including but not limited to an Electronic Compliance Indicator (ECI), which may be used to determine patient usage patterns. For example, an ECI may be configured to record sensor data from subjects (e.g., patients) wearing or intended/intending to wear an orthodontic aligner such as a shell aligner. For convenience, the examples described herein may refer to ECIs, however, it should be understood that any monitoring electronics unit may be used as described herein. The methods and apparatuses described herein may be used for any type of monitoring electronics unit, including sensors, data loggers, or the like. Unless the context makes it clear otherwise, when an “ECI” apparatus is described, the apparatus may be any monitoring electronics unit, monitoring apparatus, or performance monitoring apparatus (PMA), and are not just limited to ECI.

Some monitoring electronics unit devices, including ECIs, may include one or more sensors to detect any number of environmental characteristics. However, as some ECI devices miniaturized, their associated sensor area is also reduced. Limited sensor area may reduce the sensitivity of the ECI device. Also described herein are methods and apparatuses for increasing sensor area. These methods and apparatuses may provide surprisingly increased sensitivity with a more compact footprint then most other monitoring electronics unit.

The monitoring electronics unit (e.g., ECIs) described herein may be configured to communicate wirelessly. In particular the monitoring electronics unit may be configured to communicate through any number of near-field communication (NFC) protocols and/or NFC signals, which may transmit and receive data typically through coil antennas. The physical size of the coil antennas may affect communications and may be determined, at least in part, by the physical size of the monitoring electronics unit. Because it is often desirable to have a very small footprint for the monitoring electronics unit (in order to minimally interfere with the operation of the dental appliance and patient comfort), it may be helpful to use a very small antenna as part of the monitoring electronics unit. Unfortunately, this may result in a mismatch between the antenna of the monitoring electronics unit and the target receiving device, such as a smartphone, tablet, etc. In some cases improved communications with the ECI may be achieved by using an NFC booster; in particular, boosters that optimize the reception and transfer of data from the monitoring electronics unit. Thus also described herein are booster apparatuses and methods, including in particular NFC boosters that match and/or align NFC antennas between the monitoring electronics unit (e.g., ECI) and a target receiving device (e.g., smartphone, tablet, etc.).

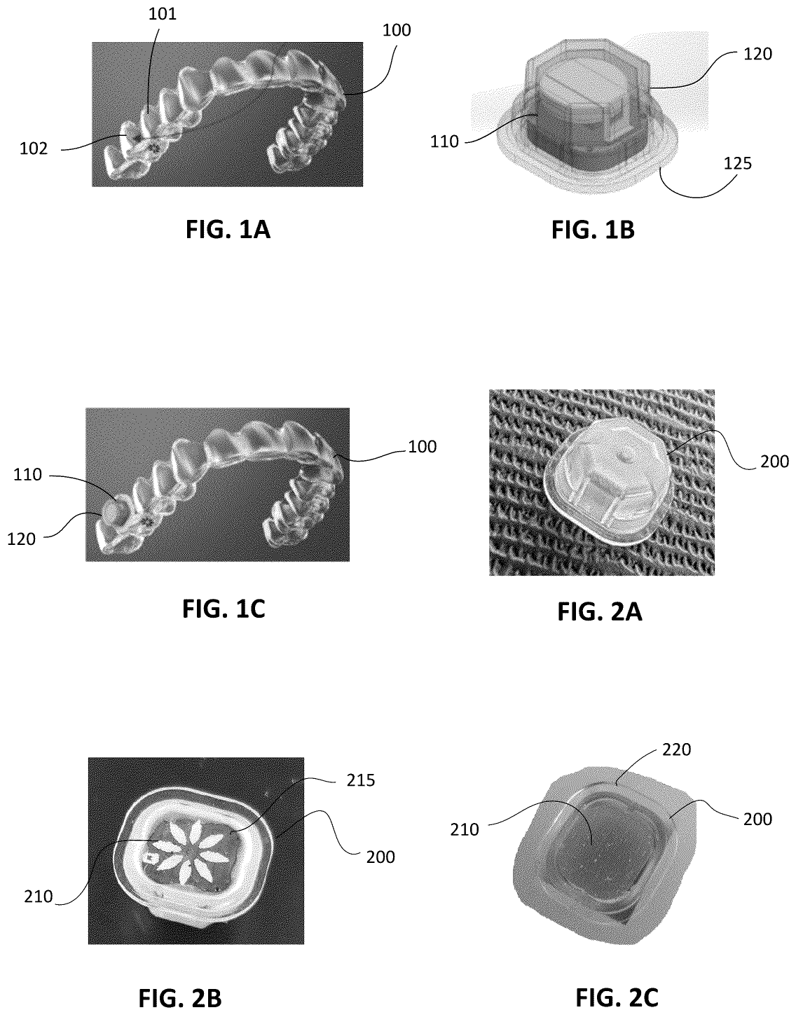

FIGS. 1A-1C show example views of a dental appliance (e.g., aligner) and a monitoring electronics unit (e.g., ECI). FIG. 1A shows an example aligner 100 that may be prepared to receive an ECI device. As shown, the aligner 100 may be associated with an upper dental arch, however, the aligner 100 may also be associated with a lower dental arch. The aligner 100 may include a number of tooth receiving cavities 101. A portion or region of the aligner 100 may be prepared to receive an ECI device mounted or disposed within a housing. Described herein are methods and apparatuses for determining and modifying a dental appliance to receive a monitoring electronics unit. For example, a region 102 of the aligner 100 may be prepared to receive and bond to an ECI housing. In some cases, the region 102 may be disposed on a tooth receiving cavity, such as tooth receiving cavity 101.

As will be described in greater detail below, the dental appliance may be configured to receive the monitoring electronics unit by forming a relatively flat mounting surface on outer surface of the dental appliance at a region that is sized, oriented and positioned on the dental appliance so to meet a number of criterion so that the monitoring electronics unit may be secured (e.g., welded) thereto without interfering with the operation of the dental appliance or the monitoring electronics unit, and without creating discomfort in the patient wearing the dental appliance, while minimizing visibility.

The mounting surface may therefore be a bonding or attaching site for the monitoring electronics unit housing, and the mounting surface may be enhanced by surface treatment of the region. In some cases, roughening, scuffing, or otherwise making the region less smooth may enhance adhesion of the housing to the aligner 100. The mounting surface (bonding region) may be substantially planar. For example, the region 102 may be shaped partially planar (e.g., flat) to form a more effective bond with the monitoring electronics unit housing.

Although shown in FIG. 1C as a buccal region of a molar, the region 102 may be any location on aligner 100 or series of aligners, as determined herein. For example, the region 102 may be lingual and/or may be associated with any feasible tooth and/or may span one or more teeth.

FIG. 1B shows a monitoring electronics unit 110 disposed within a monitoring electronics unit housing 120. The monitoring electronics unit 110 may be any feasible device such as those described at least in U.S. Pat. No. 10,470,847, which is incorporated by reference herein in its entirety. As mentioned, the monitoring electronics unit may be an ECI device that may monitor and log (e.g., record) patient use of devices, including intraoral devices such as the aligner 100. In some examples, the monitoring electronics unit housing 120 may include a mounting flange 125. The mounting flange 125 may also be shaped at least partially planar to correspond to the region 102 of the aligner 100.

FIG. 1C shows the housing 120 affixed to the aligner 100. In some examples, the housing 120 may be attached to the aligner 100 with a laser weld. As shown, the ECI device 110 may be enclosed by the housing 120 and the aligner 100. In some examples, the ECI device 110 may detect and/or log capacitance and/or temperature changes that may correspond to a patient wearing or using the aligner 100. For example, the ECI device 110 may include one or more sensors that can determine and log capacitance changes or temperature changes associated with detecting a nearby tooth, such as a tooth in an adjacent tooth receiving cavity.

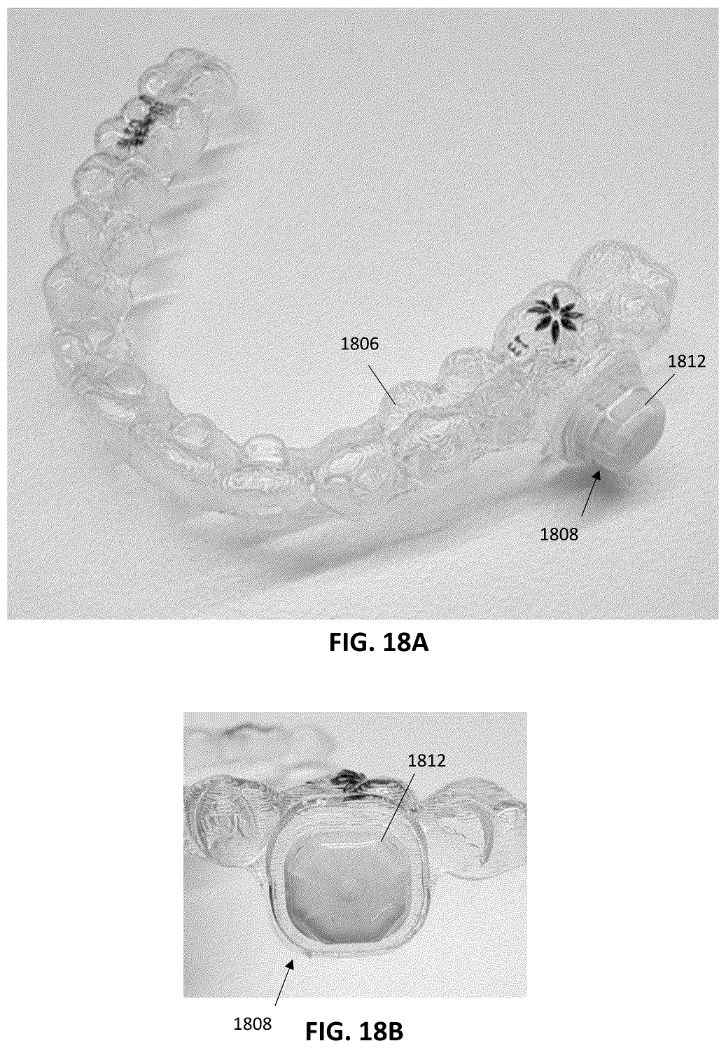

In general, the housing enclosing (partially or completely enclosing) a monitoring electronics unit (e.g., ECI device) as shown in FIG. 1C, and FIGS. 18A-18B, may be attached to the aligner in any appropriate manner, including, but not limited to laser welding, ultrasonic welding and/or a biocompatible adhesive.

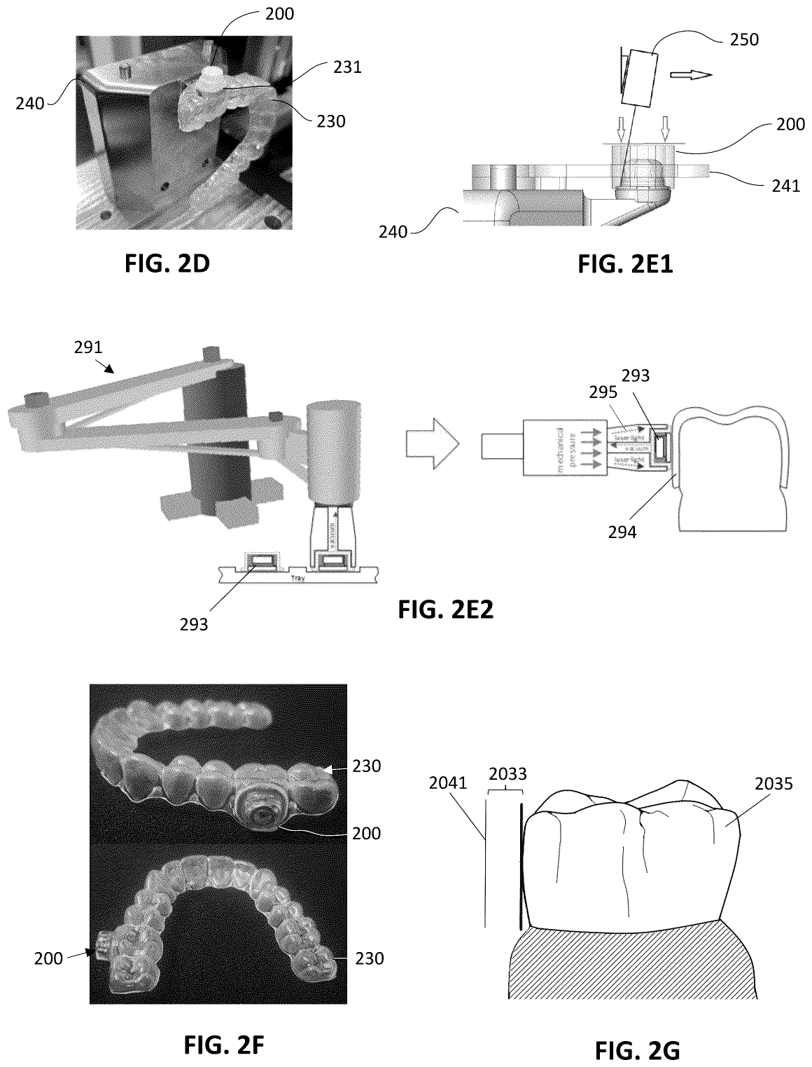

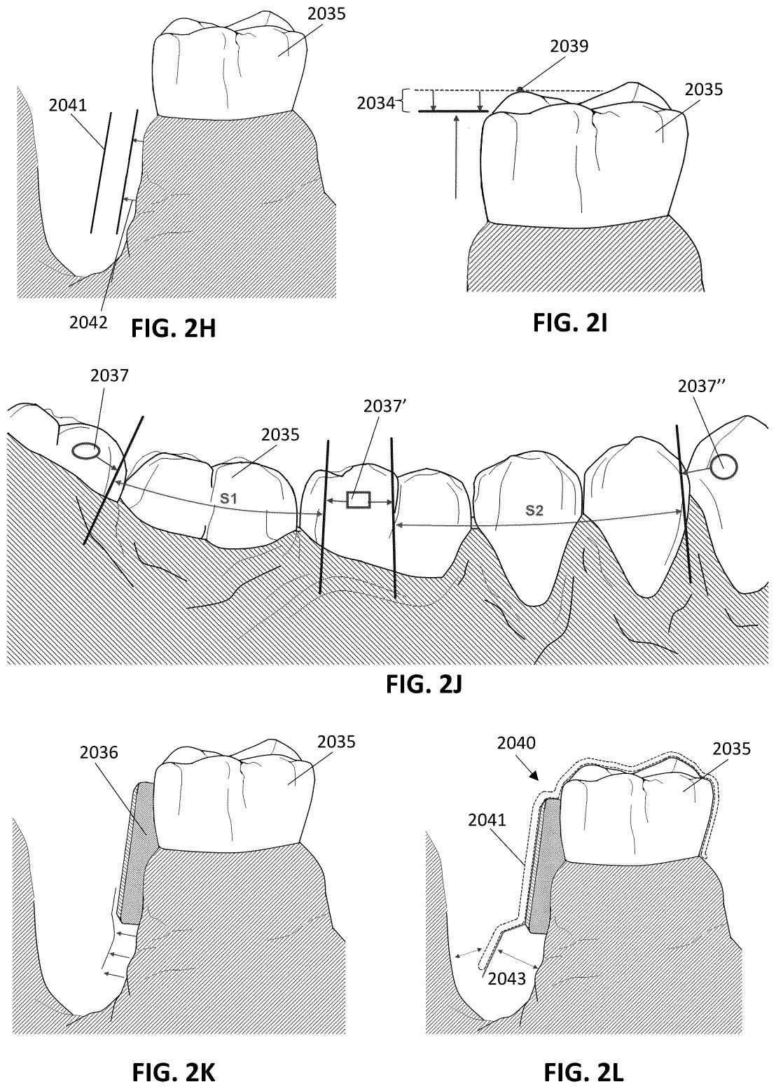

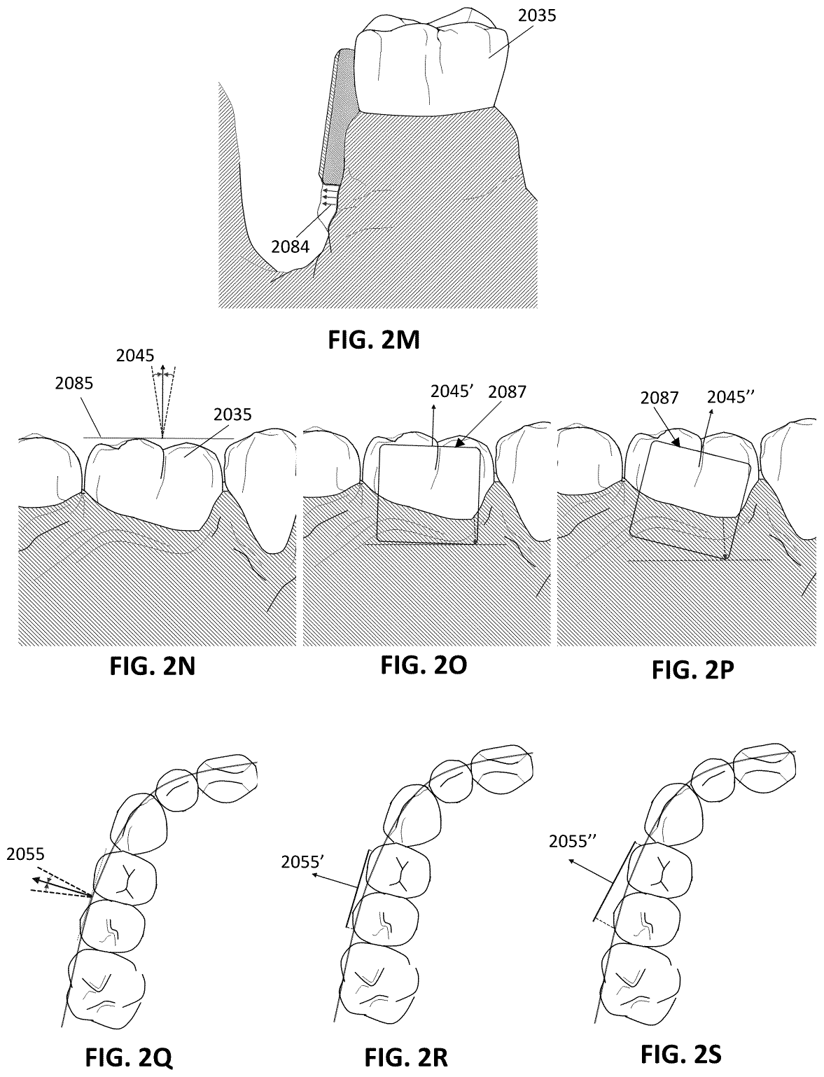

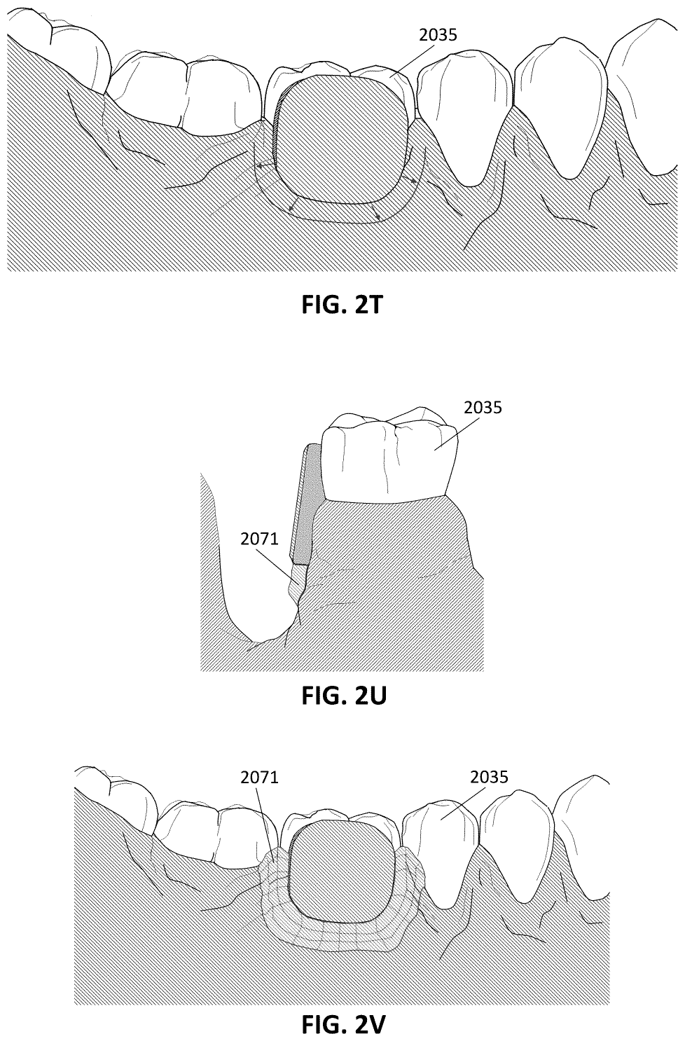

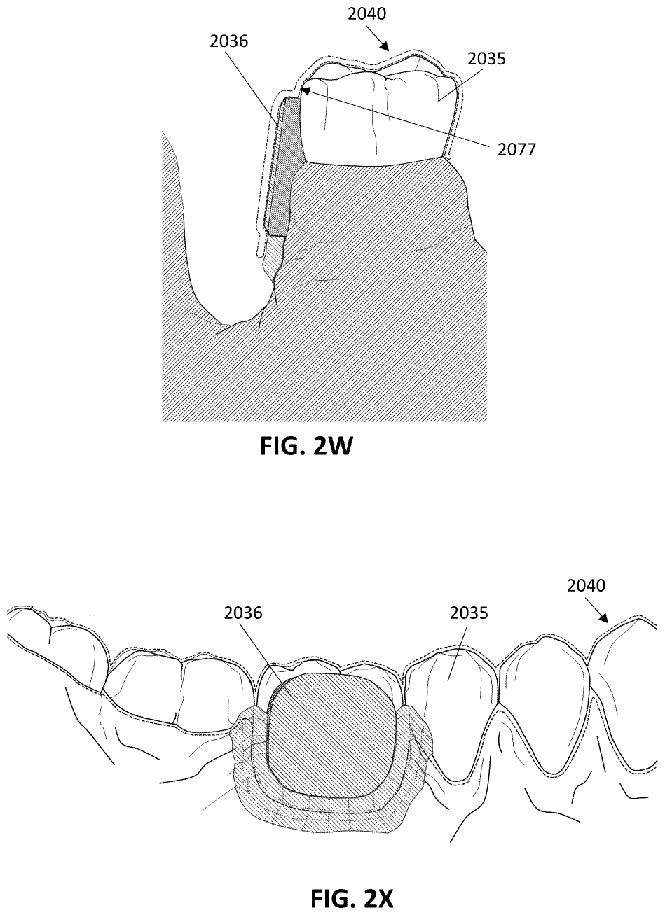

FIGS. 2A-2F illustrate some example steps for attaching an ECI device to an aligner having a mounting surface for attachment. FIG. 2A shows an example housing 200 that may be used to contain an ECI device. The housing may be similar to the housing 120 of FIG. 1. The housing 200 may be shaped to contain the monitoring electronics unit (e.g., ECI device). In some examples, the housing 200 may be shaped such that the orientation of the ECI device may be substantially controlled. In other words, the housing 200 may be shaped to receive the ECI device in a manner that positions any related sensor in a direction (e.g., toward) the patient's teeth.

FIG. 2B shows an ECI device 210 disposed in the housing 200. In some examples, the ECI device 210 may be “potted” within the housing 200. The terms potted or potting may refer to any feasible encapsulation of the ECI device 210 within the housing 200. The potting material may offer protection to the ECI device 210 as well as stabilize and affix the ECI device 210 to the housing 200. One or more electrodes 215 may be disposed on or near the surface of the ECI device 210. In some examples, the potting may not substantially affect the functionality or operation of the electrodes 215. FIGS. 18A and 18B show another example of an apparatus in which the ECI device 1808 attached to the aligner 1806 is shown fully encapsulated on all sides by an opaque potting material 1812.

FIG. 2C shows another view of the ECI device 210 in the housing 200. The housing 200 may include a mounting flange 220. The mounting flange 220 may include a treatment to enhance bonding to an aligner. For example, a surface of the mounting flange 220 may be treated with an infrared-absorbent spray (such as an infrared-absorbent clearweld spray). The infrared-absorbent spray may enhance energy absorption from laser light, including laser light having about a 1 micrometer (um) wavelength. In some cases, the infrared-absorbent spray may enhance energy absorption in any other feasible wavelength. In some other examples, the surface of the mounting flange 220 may be treated so that the infrared-absorbent spray spreads and evenly coats the mounting flange 220. In some examples, the infrared-absorbent spray may have a tint that may be visible against the housing 200, in particular when the housing 200 is clear or transparent.

FIG. 2D shows the housing 200 positioned on an aligner 230 and being held on a fixture 240. As described with respect to FIG. 1, the housing 200 may be placed on a prepared region 231 of the aligner 230. For example, the region 231 may be prepared to be substantially planar (e.g., flat) to effectively bond with the housing 200. In some examples, a fixture 240 may be used to hold the housing 200 and the aligner 230 (or any other suitable dental appliance) to assist in mounting the housing 200 on the aligner 230.

FIG. 2E1 shows a side view of the housing 200 and the fixture 240. An actuator 241 may be used in conjunction with the fixture 240 to hold the housing 200 in contact with the aligner 230 (not shown). In some examples, the actuator 241 may mechanically or pneumatically provide a clamping force to help ensure intimate contact and/or heat transfer between the housing 200 and the aligner 230. In some examples, the heat may be provided by a laser 250. The associated laser beam may be shaped as a spot, line, or field beam that may be focused on a welding surface, such as the mounting flange 220. In some examples, the laser 250 may be an Yttrium-Aluminum Garnet (YAG) laser.

FIG. 2E2 illustrates another example of welding a mounting electronics unit (e.g., ECI) to a dental appliance. The mounting electronics unit 293 may include or may be placed within a housing, as descried above, and may be poisoned, e.g., using a robotic system 291 that grasps or grabs the mounting electronics unit 293 and positions it against the dental appliance 294. This may be done using a vacuum that may hold or secure the mounting electronics unit so that it may be welded to the dental appliance, e.g., using a laser 295. The robotic arm may apply a mechanical pressure holding the housing of the mounting electronics unit against the dental appliance while simultaneously applying suction (vacuum) to securely grasp the housing.

FIG. 2F shows the housing 200 attached to the aligner 230 in a perspective view (top) and a bottom view (bottom).

The location of the mounting surface for bonding the monitoring electronics unit may be selected and prepared according to a variety of conditions so that the presence of the monitoring electronics unit on the dental appliance does not adversely affect the patient treatment. These conditions may include avoiding interactions with clinical features that are directly involved in dental/orthodontic treatment (e.g., attachments, mandibular advancement features (MAFs), precision cuts, etc), minimizing visibility of the monitoring electronics unit when worn, and maximizing comfort of the orthodontic appliance with the monitoring electronics unit when worn. It may be particularly difficult to achieve all of these conditions for a particular set of dental appliances. Described herein are methods and apparatuses (e.g., software, firmware and/or hardware) for determining an optimized location for the required monitoring electronics unit mounting surface (bonding site).

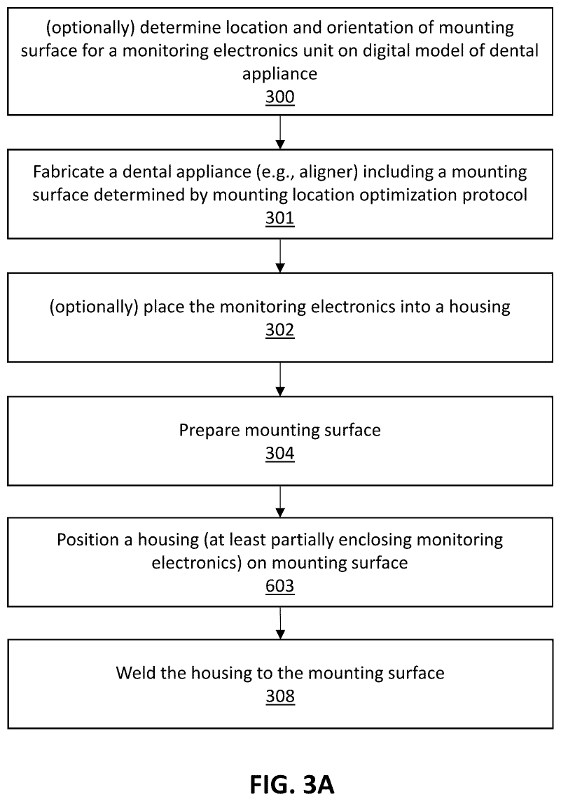

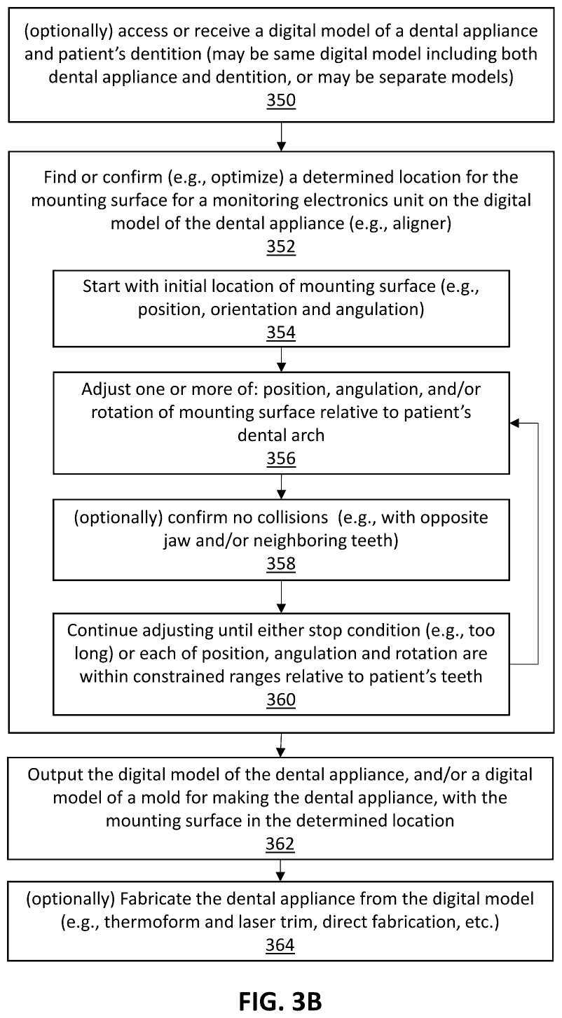

FIGS. 3A-3B illustrate examples of methods for forming an aligner including a mounting surface onto which a monitoring electronics unit can be mounted. For example FIG. 3A is a flowchart showing an example method for attaching the mounting electronics unit (e.g., ECI) to a dental appliance (e.g., aligner). Some examples may perform the operations described herein with additional operations, fewer operations, operations in a different order, operations in parallel, and some operations differently. The methods are described below with respect to a housing 200, aligner 230, and other elements illustrated in FIGS. 2A-2X, however, the method may be performed by any other suitable system, apparatus, or device.

The method shown may optionally begin by determining the location and orientation of a mounting surface for a monitoring electronics unit on a digital model of a dental appliance 300 (see also all or a part of the method shown in FIG. 3B). The method may include fabricating the dental appliance (e.g., aligner) including a mounting surface 301. Optionally, the method may include placing the electronic monitoring device into a housing 200. Some examples of an electronic monitoring device may include the ECI device 210. In some examples, the loading of the electronic monitoring device may include potting or encapsulating the monitoring device in the housing 200. The potting or encapsulating may prevent movement of the electronic monitoring device as well as provide waterproofing and protection from foreign bodies and/or tampering.

Next, in block 304 mounting surfaces on the housing 200 and/or the dental appliance are prepared. For example, the mounting flange 220 and/or the aligner 230 may receive an infrared-absorbent spray. In another example, the mounting flange 220 and/or the aligner 230 may receive a surface treatment so that an applied infrared-absorbent spray spreads evenly (e.g., does not bead).

Next, in block 306 the housing 200 is positioned on the dental appliance. In some examples, the housing 200 and the dental appliance may be positioned with respect to each other with the fixture 240 and/or the actuator 241.

Next, in block 308 the housing 200 is attached to the aligner 230. In some examples, the laser 250 may generate heat to weld or otherwise attach the mounting flange 220 to the aligner 230. Any suitable methods and systems may be used to position the housing and the aligner and to hold them in place during the attachment (e.g., welding) process, including the methods and systems described above.

In any of the monitoring electronics units (e.g., ECI's) described herein, one or more of the sensors may be include one or more components of the battery. For example, an endcap of the battery (e.g., anode or cathode) may be used as a capacitive electrode (either the anode or cathode of the battery). This configuration may save space and may take advantage of the relatively large area of the endcaps of the batteries that may be included as part of the monitoring electronics unit. In general, the relatively small and compact size of the monitoring electronics units described herein typically require that the electrodes be relatively small. By using one of the battery endcaps as one of the sensing electrodes (e.g., for capacitive sensing), the monitoring electronics unit may avoid the necessity for an additional sensing electrode.

FIG. 3B illustrates one example of a method of forming a digital model of one (or in some cases a family of related) dental appliances including a mounting surface for attaching a monitoring electronics unit. In FIG. 3B the method may optionally start by accessing or receiving a digital model of a dental appliance and a patient's dentition (step 350). In some cases, a single digital model may include both the digital model of the patient's dentition and the digital model of the dental appliance (e.g., aligner). The method may include finding or confirming (e.g., optimizing) the location for the mounting surface for a monitoring electronics unit (step 352) by iteratively adjusting one or more of: position, angulation, and/or orientation of the putative mounting surface relative to the patient's dental arch. The angulation may refer to the angle of the plane of the mounting surface relative to the patient's dentition. The position may refer to where on the dental appliance the mounting surface is positioned (e.g., over which teeth and/or which region of the teeth relative to the patient's dentition). The orientation may refer to the rotation (e.g., rotational orientation) in the plane of the mounting surface relative to the patient's dentition. The method may start with a prior starting point (e.g., position, angulation and orientation) set by the user or preset value(s) for each of the position, orientation and angulation (sub-step 354). One or more of the position, angulation, and/or orientation of the putative mounting surface relative to the patient's dental arch may then be adjusted (sub-step 356). This process may include confirming that collisions (e.g., collisions between the putative mounting surface and the opposite jaw, gingiva, and/or neighboring teeth) are not occurring by the adjustment (sub-step 358). Alternatively or additionally, the method or apparatus may check for collisions after iterating to determine a position, angulation and orientation; if collisions are detected, the process may be restarted with a different initial location 354.

The process may continue iteratively until either a stop condition is met (e.g., too many cycles, too long, too much bandwidth, etc.) or until each of position, angulation and orientation are within constrained ranges (e.g., ranges prescribed by the disclosed system as being acceptable) relative to patient's teeth (sub-step 360). Once the final (or approximate) location is determined, a digital model of the dental appliance, or a mold for making the dental appliance, including the mounting surface at the optimized position may be output, sending to a dentist/orthodontist, exporting as a file) (step 362). In some cases, the method may include fabricating the dental appliance using outputted digital model (step 364), e.g., by thermoforming, by direct fabrication, etc.

As mentioned above, FIGS. 2D-2E2 illustrate examples of ways in which these dental appliances are fabricated, including forming a mounting site. As just discussed, in some examples the mounting site may be determined so that it does not inhibit the patient's treatment. For example, the location of the mounting site may be chosen to avoid interactions with clinical features that are directly involved in a dental treatment, like attachments, MAF, precision cuts, etc. In addition, the location may be chosen to minimize visibility of the monitoring electronics unit.

As described herein, a dental appliance may be modified to include a mounting surface for a monitoring electronics unit. In general, the modified dental appliance may retain the same clinical characteristics (i.e., the modification may not affect the treatment mechanism of the dental appliance itself). However, the monitoring electronics unit improves doctors' and patients' experience with the dental treatment, and may further improve the treatment outcome by providing data that can be used to provide feedback or adjust the treatment (e.g., in real-time). In some examples, the dental appliance (e.g., aligner) may include an indicator that allows tracking of the wearing time of aligner. The disclosed system may use this “wear data” to determine that a patient is not wearing a dental appliance when the patient is supposed to (or that the patient has not worn a dental appliance for a period of time exceeding a predetermined threshold). The system may, based on this determination, send a notification via an interface (e.g., on a computer, phone, wearable device) to the patient to wear the dental appliance. The dental appliance may be modified to satisfy the conditions of the current welding process and also to adhere to one or more design constraints. The design constraints may include dimensioning to reduce patient's discomfort near the gingiva, non-interference with clinical features such as attachments, minimizing visibility (e.g., by positioning the mounting surface along one or more posterior molars), etc.

In general, the methods and apparatuses described herein may determine the location of the mounting surface for the monitoring electronics unit (e.g., ECI). The mounting surface may be flat, e.g., may include a flat area of certain size that is defined by a size and/or shape of the monitoring electronics unit. Furthermore, as described in further detail below, the mounting surface on the dental appliance may be spaced apart or angled from the teeth/gingiva (e.g., for patient comfort and safety). In the digital model, this spacing may be accomplished in some embodiments by insertion of a spacer (e.g., a “digital spacer”) which may be a digital element that is included to create a space between the dental appliance and the teeth/gingiva (e.g., referencing FIG. 2K, the digital spacer 2036).

In some embodiments, the dental appliance may be fabricated by a thermoforming process, where a mold is first fabricated (e.g., by 3D printing or any other suitable means) and a material is then thermoformed over the mold. In such embodiments, the modified digital model may be a model of the mold to be used in such fabrication. The modified digital model may be generated by generating a digital model of a mold for a dental appliance (e.g., a dental appliance such as an aligner that is prescribed for the patient based on a treatment plan) with the mounting surface in its determined location (e.g., the final location determined after the iterative process of the sub-steps of step 352 of FIG. 3B). The modified digital model of this mold may include a digital spacer and the mold that is fabricated from this modified digital model may include a physical spacer corresponding to the digital spacer. Thus, when a dental appliance (e.g., an aligner) is thermoformed over the mold (which includes the physical spacer), the dental appliance will include a spacing due the presence of the physical spacer. The physical spacer may either be formed as part of the mold, or it may be printed separately and subsequently attached to the mold in the appropriate location.

In other examples, the dental appliance (e.g., aligner) may be fabricated directly, e.g., using a 3D printing process. The modified digital model may be a model of the dental appliance itself. A spacer (e.g., digital spacer) may optionally be formed to aid in construction of such modified digital model as an intermediary step, but the digital spacer may subsequently be removed such that it is not in the modified digital model that is ultimately output. Thus, the modified digital model in these embodiments may correspond directly to the desired dental appliance and can be fabricated (e.g., 3D printed) directly using the modified digital model. The resulting dental appliance may include a space corresponding to the location of the digital spacer. Although the digital spacer may facilitate the generation of a modified digital model, it is not required, particularly when the dental appliance is being fabricated directly from the modified digital model (as opposed to an indirect method like thermoforming). In some examples, the separate construction of a digital spacer is not required, and an initial model of a prescribed dental appliance may simply be modified directly without creating a digital spacer. In some embodiments, the modified digital model may be sent to a fabrication system (e.g., a 3D printing system) for directly fabricating the dental appliance with the mounting surface.

The mounting surface may be positioned on a buccal surface of the dental appliance. Mounting (e.g., welding) the monitoring electronics unit on a lingual part potentially can significantly reduce patient comfort as the indicator protrudes above the aligner surface and may disturb the patient's tongue. In addition, it can be dangerous if a patient accidentally bit the indicator and broke aligner, the patient may hurt themselves or even swallow the indicator. Thus, the buccal surface of the dental appliance may be considered as a safer option for indicator welding. The dental appliance may still have to fit tightly to the patient's teeth as the dental appliance(s) move the teeth towards the planned directions. To satisfy this condition, the top surface of the mounting surface may be located as close to the teeth surface as possible. However, in some cases, the mounting surface may be positioned on a lingual surface, particularly in cases where the buccal surface is not an option (e.g., when doing so would interfere with attachments or a treatment mechanism). For example, the dental appliance may be a palatal expander that has buccal sides extending only over a subset of molars and one or more of these molars may have retention attachments on the buccal sides. As such, there may not be sufficient room to mount a monitoring electronics unit on the buccal side. In such cases, the mounting surface may be placed on the lingual side. More information about palatal expanders with retention attachments may be found in U.S. Pat. No. 11,273,011 (“Palatal expanders and methods of expanding a palate”), which is incorporated by reference herein in its entirety.

As mentioned above, these methods of optimizing the position of the mounting surface may include iterating until the position, the angulation and/or the orientation of the mounting surface in the digital model of the dental appliance are within a constraint range relative to the digital model of the patient's dentition, and as long as the mounting surface does not collide with the patient's teeth or with a treatment feature of the aligner. For example, FIG. 2G shows an example of adjusting a distance 2033 from the mounting surface 2041 to a tooth 2035 on its buccal surface (i.e., left side of the tooth 2035 in FIG. 2G). In some examples the constraint range of the distance from the mounting surface to the tooth buccal surface is between about 0.2 and 1.0 mm.

FIG. 2H shows an example of maintaining a safety gap between the mounting surface 2041 and the gingiva along a portion of the mounting surface 2041 that may extend over the gingiva in some examples. If the mounting surface were allowed to contact the gingiva, this may cause patient discomfort and in some cases may lead to health issues, particularly in cases where the dental appliance is worn for extended periods (e.g., extended pressure or contact with soft tissue such as the gingiva can cause lesions, tissue damage, and/or infections). In some examples, the dental appliance may be configured to include a safety gap 2042 between the dental appliance and the gingiva to prevent or reduce contact. Thus, the dental appliance may be configured so that the appliance maintains a distance from the mounting surface to the tooth, as well as the safety gap between the dental appliance and the gingiva. Similarly, FIG. 2H shows an example of a region 2084 that is offset from the gingiva; the mounting surface or an extension of the mounting surface (e.g., a buffer region) may be kept separated from the gingiva by this minimum distance (e.g., safety gap). Because the mounting surface may be relatively flat, while the gingiva and tooth surface underlying the mounting surface portion of the dental appliance may be variable in height, the inner surface of the dental appliance, opposite from of the mounting surface, may be spaced apart (by the iterative process described above), so that the minimum distance between the gingiva and the inner surface of the dental appliance, opposite from of the mounting surface is between the constrained range of the safety gap (e.g., between about 1 mm and about 0.5 mm, between about 0.75 mm and about 0.5 mm, between about 1 mm and 0.4 mm, between about 0.8 mm and 0.4 mm, between about 1 mm and about 0.4 mm, etc.).

For example, in order to ensure comfort and safety, the safety gap may be at or greater than a minimum permissible distance. However, a further constraint is that the mounting surface may not extended too far away from gingiva, which may irritate the adjacent tissue and may interfere with the fit of the dental appliance. Thus, the safety gap must be within a predetermined safety gap range, with a minimum permissible distance and a maximum permissible distance from the gingiva being predetermined. In some examples, the safety gap may be between about 1 mm and 0.5 mm. In some cases, this safety gap range may be uniquely determined for the patient based on, e.g., a scan or photos of the patient's intraoral cavity. In some embodiments, the actual gap for a patient is determined with an iterative process as described above, in which the position, angulation and/or orientation of the mounting surface is adjusted until the gap between the mounting surface and the gap between the aligner and the gingiva is within the predetermined safety gap range from the gingiva along the portion of the dental appliance opposite from the mounting surface.

FIG. 2I illustrates constraints for vertical positioning of the mounting surface. In some embodiments, the mounting surface location may be further optimized by identifying a position that minimize the extension over the gingiva (e.g., to minimize the possibility of discomfort from potential gingival contact), while avoiding interference with the patient's bite. This may be done by positioning the mounting surface as close as permissible to the occlusal surface. Permissibility in this context may be determined based on a risk of a mounted monitoring electronics unit interfering with the patient's bite/occlusion. Otherwise, the monitoring electronics unit may break or become dislodged and/or the monitoring electronics unit may cause patient discomfort. Thus, in configuring the mounting surface, the vertical positioning of the mounting surface may be constrained so that it does not exceed within a predetermined distance from the incisal points of one or more teeth (e.g., the incisal points of the tooth to which the monitoring electronics unit will be mounted), or within a region that may interfere with teeth from the opposite jaw when the jaws are closed. In some embodiments, when locating the mounting surface, a vertical safety distance 2034 may be imposed such that any point on the mounting surface (in FIG. 2I, the top of the mounting surface) is distanced away from the lowest incisal point 2039 of the tooth 2035 to which the monitoring electronics unit will be mounted. In some examples, the vertical safety distance may be between a permissible range of within about 1 mm. This vertical safety distance (e.g., an incisal clearance) may allow the monitoring electronics unit to be positioned as close to the occlusal surface (and away from the gingiva) while avoiding bite interference. This vertical safety distance may also enhance retention. For example, increasing the vertical safety distance may allow the dental appliance to contact the upper buccal side of the tooth or teeth if increased retention is desired. As illustrated in FIG. 2W, the dental appliance 2040 contacts (arrow 2077) the buccal side (e.g., the left side of the tooth 2035 in FIG. 2W) for a short distance before it extends outward to form the mounting surface. As described above, the actual vertical distance between the upper edge of the mounting surface for a particular patient's dental appliance may be determined by an iterative process that adjusts the position, angulation and/or orientation of the mounting surface until the upper edge of the mounting surface (measured from the lowest incisal point to the mounting surface) is between the is within the predetermined permissible range of the vertical safety distance.

In addition, these methods and apparatuses may determine a horizontal position (i.e., the position along the dental arch) of the mounting surface relative to the buccal teeth surface of the teeth by identifying which tooth or set of teeth (including regions between two or more teeth) would be optimal for mounting the monitoring electronics unit. The horizontal position of the mounting surface may be constrained so as not to collide with existing treatment features on the teeth or dental appliance. Such features may be apparent from the dental appliance, and/or the methods and apparatuses described herein may review the patient's treatment plan (which may include a 3D model of the patient's teeth with any clinical features such as attachments) to prevent such collisions. In addition, the horizontal position may be selected to minimize visibility and/or maximize comfort. In some cases the mounting surface may be preferentially positioned over a posterior molar. FIG. 2J illustrates a step in the process of determining the horizontal position. An allowed distance from the mounting surface to the treatment features may be restricted with a certain value due to the manufacturing process. In the example of FIG. 2J, there are two allowed regions, S1 and S2, for a new object to be positioned on the buccal surface of the dental appliance. These regions may be determined as allowable at least in part because they do not have any clinical features (e.g., attachments, cut lines, etc.) and are sufficiently spaced apart from clinical features 2037, 2037′, 2037″. Although S2 provides a bigger area for the placement, S1 may be chosen in this example because placing the unit on the molars (more posteriorly) may be less visible (e.g., to external observers during ordinary wear). The mounting surface can be positioned so it covers several teeth or a single tooth. As mentioned, the horizontal position of the mounting surface on the dental appliance may be determined by, iteratively adjusting the horizontal position of the mounting surface so that the distance from an edge of the mounting surface to a treatment feature 2037, 2037′, 2037″ (e.g., attachment, etc.) of the dental appliance is maintained sufficiently far (e.g., about 0.1 mm, about 0.2 mm, about 0.3 mm, about 0.4 mm about 0.5 mm or more, etc.).

FIGS. 2N-2P illustrate the constraints on the rotational orientation of the mounting surface (i.e., the orientation of the mounting surface relative to a plane through the occlusal surface of the teeth 2085 (or teeth) and thus the vertical orientation of the monitoring electronics unit when it is mounted. For example, in FIGS. 2N-2P, a normal vector from the upper edge 2087 of the mounting surface 2045 may be constrained so as not to deviate beyond a predetermined range from a normal to the occlusal plane 2085 of the tooth (or teeth) adjacent to the mounting surface. Permissible rotational orientations in this example are illustrated by the angular ranges 2045 shown in FIG. 2N. Placing the mounting surface in a vertical orientation with significant deviation (as shown in FIG. 2P, in which the normal vector 2045″ of the upper edge 2087 of the mounting surface extends beyond the predetermined constraint range from the normal of the occlusal plane of the teeth) may increase the probability of an undesirable interaction of the mounted monitoring electronics unit and teeth on the opposite jaw. Thus, the rotational constraint (e.g., restricting rotation of the mounting surface) may minimize the risk of biting the monitoring electronics unit by the patient. In addition, this may minimize the area of the mounting surface that may come into contact with the gingiva.

In some examples the horizontal (rotational) orientation of the mounting surface (i.e., the orientation of the mounting surface parallel to a plane of the occlusal surface of one or more teeth adjacent to the mounting surface) may also be constrained. This is illustrated in FIGS. 2Q-2S, which show different horizontal orientations of the mounting surface. The horizontal orientations may be constrained to limit the deviation of a normal vector of the mounting surface relative to a normal vector of a plane in the buccal surface of one or more teeth along this plane. A larger deviation may lead to non-optimized position of the mounting surface that is shown, e.g., in FIG. 2S. The angulation of the mounting surface relative to the buccal plane of the teeth over which it is to be located may be iteratively adjusted as described above, so that a normal to the mounting surface 2055, 2055′, 2055″ is within a constrained range relative to a normal to a jaw arch of the patient's dentition (e.g., the buccal plane of the tooth or teeth adjacent to the mounting surface). This constraint range for the angle between the normal of the mounting surface and the normal to a jaw arch of the patient's dentition may be between −12.5 and 12.5 degrees (e.g., between +/−15 degrees, between +/−12.5 degrees, between +/−10 degrees, between +/−8 degrees, etc.).

The restrictions may be defined as targets and constraints for identifying the best possible position and orientation of the mounting surface for each case (e.g., for a particular dental appliance for a patient, for a particular stage or set of treatment stages for a particular patient in a multi-staged dental treatment, etc.). Acceptable ranges may be defined for each metric, as mentioned above. Solving the optimization problem that includes all targets and constraints may provide a fast and reliable way for positioning the platform on patient's jaw in a treatment model. This method provides a technical solution to the technical problem posed by the need to position the monitoring electronics unit. In general, the optimization problem can be solved for each stage of a treatment plan independently.

However, in any of these methods an apparatuses, an additional constraint can be included in order to get a smoother trajectory of the mounting surface between the different dental appliances that are worn in a sequence. In this case, the problem may be solved on a range of stages (appliance) to be worn in the sequence.

As shown in FIGS. 2T-2V and 2W-2X, the appliances, including the mounting surfaces may also be configured to provide sufficient space to avoid gingiva and to allow trimming of the appliance after fabrication. For example, a dental appliance may be modified to allow trimming as part of the fabrication. The welding process may also include cutting the dental appliance with a small gap, so the area prepared for indicator welding is stayed untrimmed.

As shown in FIGS. 2K and 2L, the dental appliance 2040 (e.g., aligner) may have a safety gap between the gingiva surface and the area around the mounting surface 2041 of the dental appliance 2040. In some cases, as shown in FIGS. 2K and 2L, a digital spacer 2036 may be included in the model of the patient's teeth against which the dental appliance may be formed (e.g., thermoformed). In general, the methods for designing and making these apparatuses may avoid sharp corners on appliance edge, and may include a cut (trimming) line that is smooth, especially around the mounting surface, as it may be quite close to the patient's gingiva. The apparatus may include an inner surface 2036 that preserves a safety gap everywhere around the mounting surface relative to the gingiva. The distance from this surface to gingiva may not be less than a required safety gap to avoid aligner interaction with the gingiva, and the distance may not exceed the safety too much as in this case it could interfere the welding process of indicator or hurt the patient's cheek. In FIG. 2L, the distance 2043 between the dental appliance edge and patient's gingiva may be configured to not exceed a predetermined threshold such that the edge of the dental appliance does not hurt or excessively contact the patient's cheek.

In any of these examples and devices, the mounting surface formed on the dental appliance may be at least partially surrounded by a buffer region, that may allow trimming of the dental appliance (e.g., laser trimming) and/or may prevent interference between the mounting surface and one or more structures of the oral cavity. As shown in FIG. 2U, the region around and extending from the mounting surface may be spaced apart from the surface of the gingiva but may remain substantially parallel to the gingiva (preserving a predetermined distance to the gingiva). The additional buffer region around the mounting surface may be kept relatively small, and the size may be enough to preserve a safety gap around the mounting surface while allowing trimming along a cut or trim line. This is illustrated in FIG. 2X.

For example, FIGS. 2U and 2V illustrate a buffer region 2071 providing a gap between the mounting surface and an edge of the dental appliance that may be trimmed. Thus, a cut or trim line may be located on this buffer region some distance from the mounting surface. FIGS. 2W and 2X illustrate a modified cutting (trimming) line around the mounting surface that cuts through this buffer region.

### Sensors

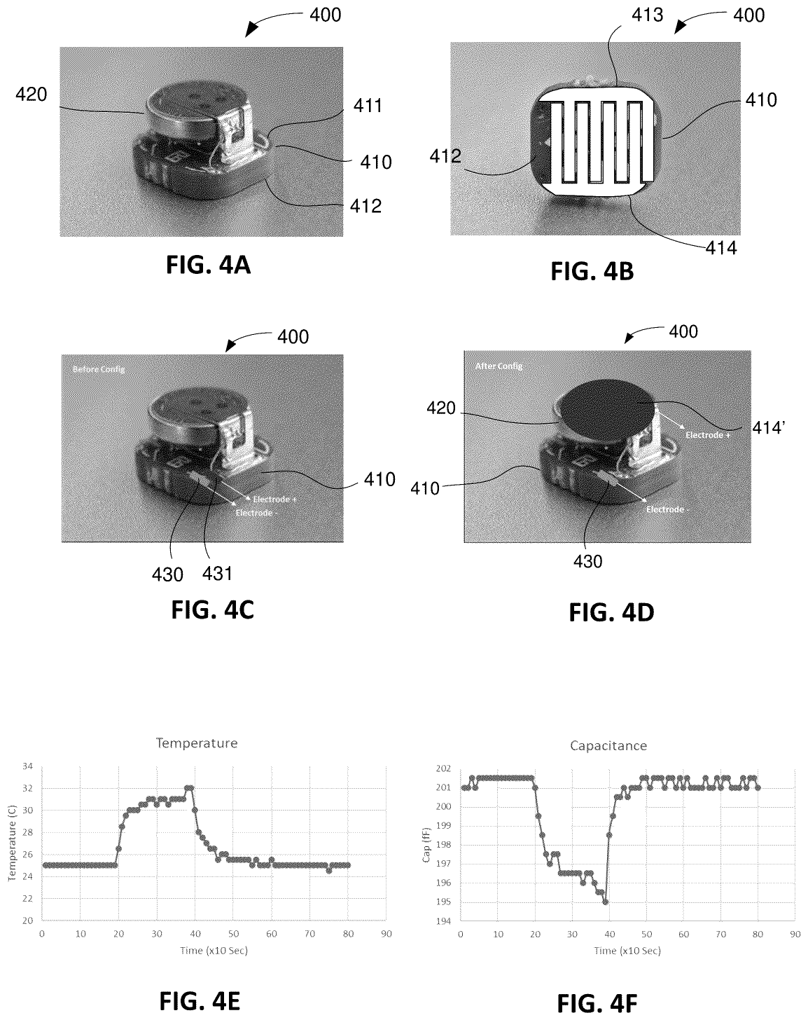

The area of a capacitance sensor may be related to the sensor sensitivity. Therefore increased sensor area may increase sensor sensitivity. Described herein are method and devices for increasing sensor sensitivity without substantially increasing (and in some cases decreasing) the size of an apparatus including the sensor are shown herein. For example a portion of a battery (e.g., the battery endcap) may be used as part of the sensor to increase sensor area. This is illustrated in FIGS. 4A-4D. FIG. 4A shows an example of an ECI 400. The ECI 400 may include a printed circuit board (PCB) 410 and a battery 420. The ECI 400 may also include one or more electronic circuits, components, or devices (on the PCB) to measure or sense an environmental characteristic. For example, some electronic circuits, components, or devices may measure and/or log capacitance and or temperature near the ECI 400. The battery 420 may power the electronic circuits, devices, and/or components. The battery 420 and the electronic circuits, components, or devices may be disposed or located on a first side 411 of the PCB 410. A second side 412 of the PCB may include sensors.

FIG. 4B shows the second side 412 of the PCB 410 of the ECI 400 of FIG. 4A. The second side 412 may include one or more electrodes. As shown, the second side 412 may include a first electrode 413 and a second electrode 414. Surface area of the first electrode 413 and the second electrode 414 may be limited, in part, by the size of the PCB 410. FIG. 4C shows example connections to the first electrode 413 and the second electrode 414 on the second side 412 of the PCB 410 of FIGS. 4A-4B. For example, a first connection 430 may couple the first electrode 413 to one or more electronic circuits and a second connection 431 may couple the second electrode 414 to one or more electronic circuits. In some examples, the first connection 430 and the second connection 431 may use one or more vias to connect to the first electrode 413 and the second electrode 414, respectively.

FIG. 4D shows an example of modified electrodes for the ECI 400 in which the upper surface of the battery (e.g., cathode) is used to form the electrode. In some examples, at least one surface of the battery 420 may be used instead of, or in addition to one of the electrodes on the bottom of the ECI 400 as shown in FIG. 4B. For example, a terminal (endcap) of the battery 420 may be used as part of the second electrode 414′. Thus, the second connection may be coupled to the positive terminal of the battery 420. In some examples, the negative terminal of the battery 420 may be used instead of the positive terminal. Thus, in some examples the battery 420 may function as a second electrode. The larger surface area provided by the case/terminal of the battery 420 may increase the sensitivity of the first electrode 413 and/or the second electrode 414. The battery 420 may advantageously provide additional sensor surface area without the addition of any additional bulky components. The electrode surface area provided by the battery may be comparable to the sum of the surface area of other components on the PCB 410.

FIGS. 4E and 4F are graphs of the sensed temperature and capacitance, respectively, using the configuration shown in FIG. 4D, which are comparable or better than measurements taken using a configuration as shown in FIG. 4B. In FIG. 4E the temperature measurement is comparable between these two configurations, while FIG. 4F shows the capacitance measured using the configuration of FIG. 4D. In general, the more compact, but larger surface area configuration of FIG. 4D, results in more robust capacitive sensing as the capacitance is measured over a larger sensing area, improving its sensitivity to detect the object in proximity to the top side of this device. In FIG. 4F, the capacitance changes are the result of a finger contacting the top side of the battery on this device.

This configuration may permit the battery to form an electrode on one side of the PCB that it covers, and the PCB can have another electrode or electrode pair built into it (e.g., on the bottom, as shown in FIG. 4B). The battery and the electrode or one of the electrode pair can then be used as one set for capacitive sensing and also be used as its original pair. This can create not only a sensor on the side of the PCB with the electrode pair, but another touch sensor on the backside of the device where the battery is located.

In some other examples, an electrode may be added to the housing 200 of FIG. 2. For example, a metal or metallic element or coating may be applied to the housing 200 to operate as an additional electrode. Such configuration may not add any bulky components and advantageously use a surface that may otherwise be unused.

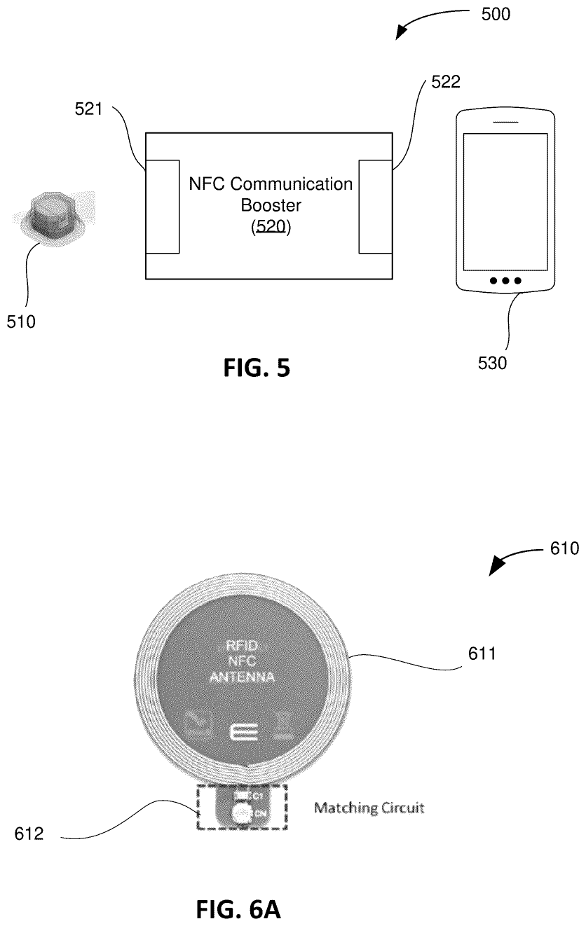

FIG. 5 is a block diagram of an example NFC system 500. The NFC system 500 may include an NFC device 510, an NFC booster 520, and a communication device 530. The NFC device 510 may include a transceiver configured to wirelessly transmit and receive data using NFC signals. In some examples, the NFC device 510 may include other circuits including sensing circuits and/or batteries. Example NFC devices 510 may include any of the ECI devices described herein, including but not limited to the ECI device 110 of FIG. 1.

The communication device 530 can be any device that can communicate via NFC signals. In some examples, the communication device 530 may include a plurality of wireless and/or wired transceivers. For example, in addition to an NFC transceiver, the communication device 530 may include Wi-Fi, Bluetooth, and/or cellular wireless transceivers. Although illustrated as a smartphone, the communication device 530 may be implemented as a mobile phone, tablet computer, laptop computer, or any other feasible device.

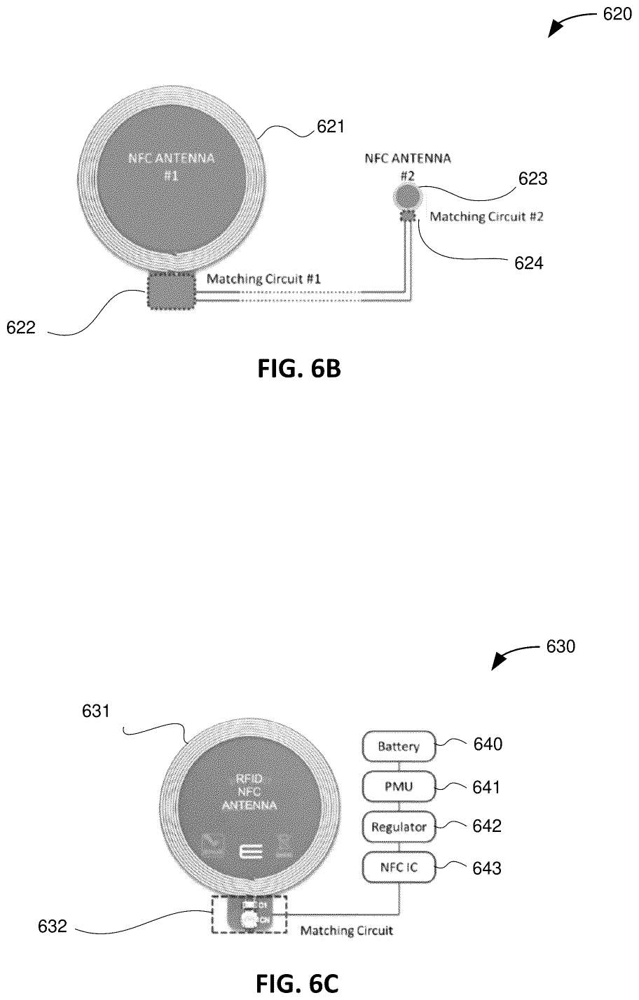

The NFC booster 520 may enable reliable and robust NFC data transfer between the NFC device 510 and the communication device 530. In some examples, the NFC device 510 may include a first antenna 521 and a second antenna 522. The first antenna 521 may be optimized for NFC communications with the NFC device 510 and the second antenna 522 may be optimized for NFC communication with the communication device 530. In some examples, NFC communications may include NFC signals having a frequency of approximately 13.56 MHz. For example, the size of the first antenna 521 may be optimized for NFC signal coupling with the NFC device 510. That is, the size of the first antenna 521 may be similar to the size of the NFC antenna in the NFC device. Similar antenna sizes may enable efficient NFC signal transfer. In a similar manner, the size of the second antenna 522 may be similar to the size of the NFC antenna in the communication device 530. The NFC booster 520 may enhance wireless communications between the NFC device 510 and the communication device 530 by advantageously processing, and in some cases amplifying, NFC signals. Example NFC booster designs are described below in conjunction with FIGS. 6A-6C.

FIGS. 6A-6C show example NFC boosters. FIG. 6A shows an example NFC booster 610. The NFC booster 610 may be a passive design (e.g., does not include a battery) that includes antennas 611 and a matching circuit 612. The antennas 611 may include a first antenna and a second antenna (first and second antennas not shown). The first antenna of the antennas 611 may transmit and receive NFC signals to and from an NFC device (such as the NFC device 510 of FIG. 5) and the second antenna of the antennas 611 may transmit and receive NFC signals to and from a communication device (such as the communication device 530). The antennas 611 may include two separate and independent multi-turn conductive coils to implement the first antenna and the second antenna. The first antenna and the second antenna may be concentric with respect to each other. The matching circuit 612 may match or adjust an apparent antenna impedance mismatch between the first antenna and the second antenna. Some impedance mismatches may be caused by differing NFC antenna sizes with between the first antenna and the second antenna.

FIG. 6B shows another example NFC booster 620. The NFC booster 620 may be a passive design that includes a first antenna 621, a first matching circuit 622, a second antenna 623, and a second matching circuit 624. The first antenna 621 may be larger than the second antenna 623. In some examples, the first antenna 621 may transmit and receive NFC signals to and from the communication device. Thus, the first antenna 621 may have a diameter similar to an NFC antenna within the communication device. In some examples, the first antenna may have a diameter of about five centimeters (cm). Similarly, the second antenna 623 may transmit and receive NFC signals to and from the NFC device. Therefore, in some examples, the second antenna 623 may have a diameter of about one cm. The first matching circuit 622 and the second matching circuit 624 may match impedances between the first antenna 621 and the second antenna 623.

FIG. 6C shows another example NFC booster 630. The NFC booster 630 may be an active design (e.g., includes a battery) that includes antennas 631 and a matching circuit 632. Similar to the antennas 611 of FIG. 6A, the antennas 631 may include a first antenna and a second antenna (individual antennas not shown). The first and second antennas may be concentric with respect to each other. Similar to the matching circuit 612, the matching circuit 632 may match or adjust the apparent antenna impedance mismatch between the first and second antennas. The matching circuit 632 may include a variety of electronic circuits or devices. For example, the matching circuit 632 may include a battery 640, a power management unit 641, a voltage regulator 642, and an NFC integrated circuit 643.

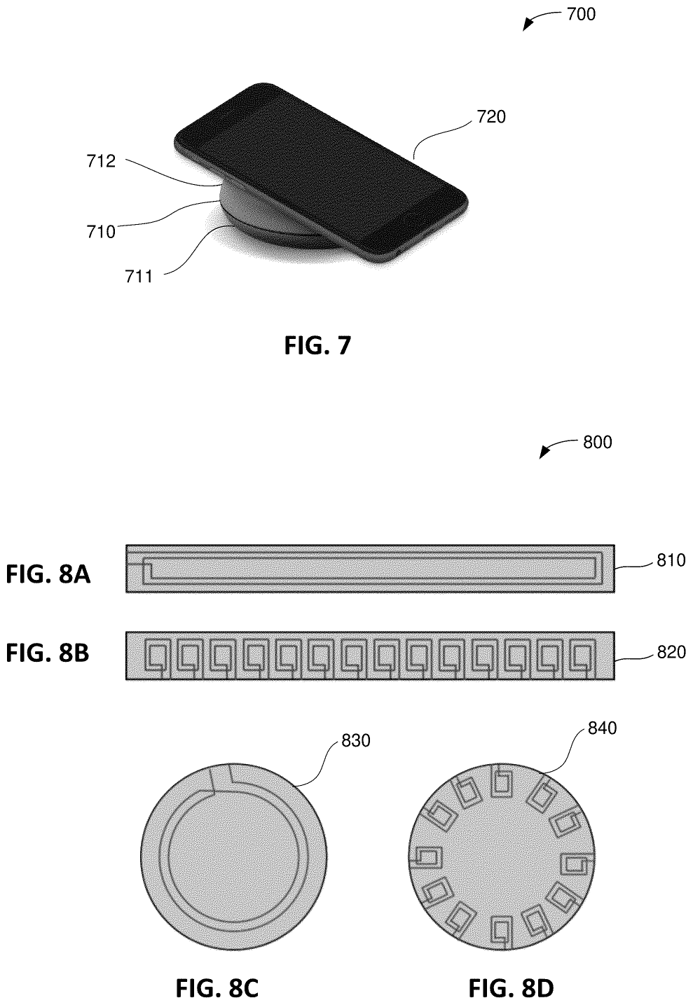

FIG. 7 shows an example NFC system 700. The NFC system 700 may include a case 710 and a communication device 720. The case 710 may include a base 711 and a lid 712. The lid 712 may be movably coupled to the base 711. The case 710 may be used to store or contain an aligner with an attached NFC device. For example, the case 710 may include an inner chamber to store any of the aligners and ECI devices shown in FIGS. 2A-2F. In some examples, the case 710 may include an NFC booster such as any of the NFC boosters shown and described in FIG. 6A-6C. In some examples, the lid of the case 710 may be shaped to support (e.g., receive) the communication device 720. In some other examples, the case 710 may be placed in contact with a screen of the communication device 720. For example, the case 710 may be placed on the screen of the communication device 720.

In some examples, the lid 712 of the case 710 may include an antenna to transmit and receive NFC signals to and from the communication device 720. In some examples, the lid 712 of the case 710 may also include an antenna to transmit and receive NFC signals to and from the NFC device.

In some examples, when an NFC device is placed in an inner chamber of the case 710, and the communication device 720 is brought near the case 710, data may automatically be transferred between the NFC device and the communication device 720.

FIGS. 8A-8D shows example antenna designs 800 that may be used with a case. In some examples, the case may be an example of the case 710 of FIG. 7. Thus, the case may include a first antenna, a second antenna, and an NFC booster. Any of the antenna designs described herein may be used and coupled to the NFC booster. A first antenna assembly 810 (FIG. 8A) may be placed on an inner circumference of the inner chamber of the case. The first antenna assembly 810 may transmit and receive NFC signals to and from an enclosed NFC device. The first antenna assembly 810 may include a multi-turn coil of conductive material that may effectively traverse substantially all the inner circumference. The conductive material can be copper wire, conductive tape, a polymer treated with a conductive paint, conductive glue, or the like.

A second antenna assembly 820 (FIG. 8B) may include a plurality of multi-turn coils of conductive material. In some examples, the size of one of the multi-turn coils may be comparable to the size of the NFC antenna of the NFC device. In some examples, the multi-turn coil nearest to the NFC device may be selected to receive and transmit NFC signals. That is, a particular multi-turn coil may be selectively coupled to the NFC booster. Using the multi-turn coil that is closer to the antenna size of the NFC device and/or physically closer to the NFC device may advantageously improve NFC data transfer.

A third antenna assembly 830 (FIG. 8C) may be affixed to a lid or a bottom of the case. Similar to the first antenna assembly 810, the third antenna assembly may include a multi-turn coil of conductive material. In some examples, the third antenna may be positioned over possible locations of the NFC device within the inner chamber.

A fourth antenna assembly 840 (FIG. 8D) may also be affixed to a lid or bottom of the case Similar to the second antenna assembly 820, the fourth antenna assembly 840 may include a plurality of multi-turn coils of conductive material. In some examples, the multi-turn coil nearest to the NFC device may be selected to receive and transmit NFC signals to and from an NFC device within the inner chamber.

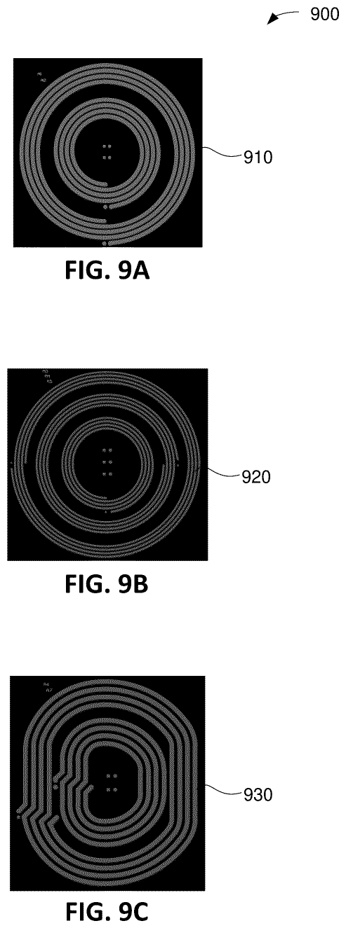

FIGS. 9A-9C show examples of multi-coil antenna designs 900. The antenna designs 900 may be used with any case, such as the case 710 of FIG. 7. Alternatively, or in addition, the antenna designs 900 may be used with any of the NFC boosters described herein. A first antenna design 910 may include two circular-shaped multi-turn coils that are concentric with each other. A second antenna design 920 may include three circular-shaped multi-turn coils that are concentric with each other. A third antenna design 930 may include two oval-shaped multi-turn coils that are concentric with each other. Any of these antenna designs 910-930 may be used with any NFC booster described herein. In some examples, any of the antenna designs 910-930 may be disposed or mounted on a lid or bottom of a case.

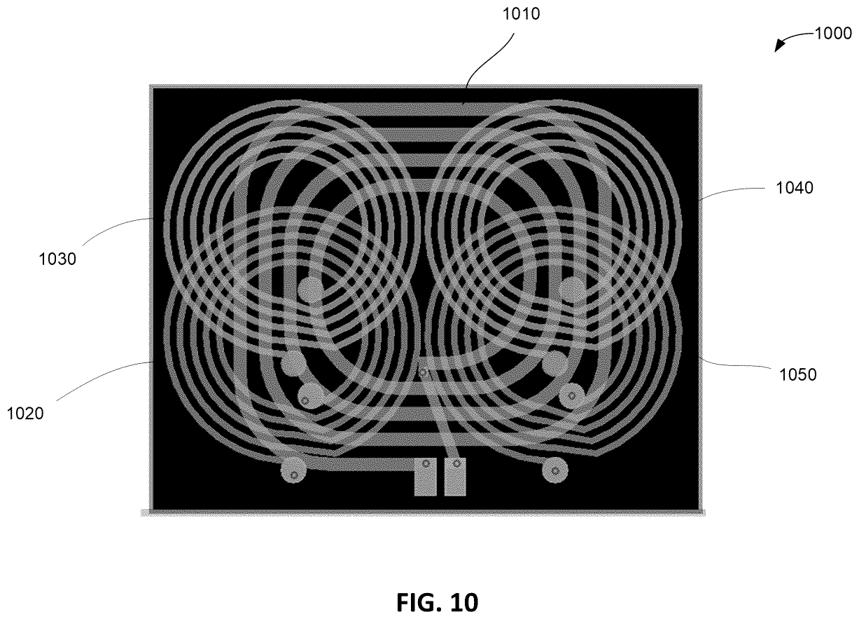

FIG. 10 shows another example antenna assembly 1000. The antenna assembly 1000 may be used with any case and/or any NFC booster described herein. The antenna assembly 1000 may include a first antenna 1010, a second antenna 1020, a third antenna 1030, a fourth antenna 1040, and a fifth antenna 1050. Although only five antennas are shown in FIG. 10, in other examples, the antenna assembly 1000 may include four or fewer antennas. In some other examples, the antenna assembly 1000 may include six or more antennas.

The first antenna 1010 may include a multi-turn coil of conductive material. As shown, the first antenna 1010 may have generally a square shape with substantially rounded corners. In other examples, the first antenna 1010 may have any other feasible shape. The second antenna 1020, the third antenna 1030, the fourth antenna 1040, and the fifth antenna 1050 may each include a multi-turn coil of conductive material and have a circular shape. In other examples, the second antenna 1020, the third antenna 1030, the fourth antenna 1040, and the fifth antenna 1050 may have any other feasible shape. In some examples, one antenna may partially or completely overlap one or more other antennas.

Each antenna in the antenna assembly 1000 may be used individually or in combination with each other. Thus, two or more antennas may be coupled simultaneously to an NFC booster and operate as a single antenna. In some examples, the use of a combination of two or more antennas may provide increased NFC performance, especially compared to using a single antenna. Any combination of antennas may be used. For example, the first antenna 1010 may be used in combination with the second antenna 1020. In another example, the second antenna 1020 may be used in combination with the fourth antenna 1040.

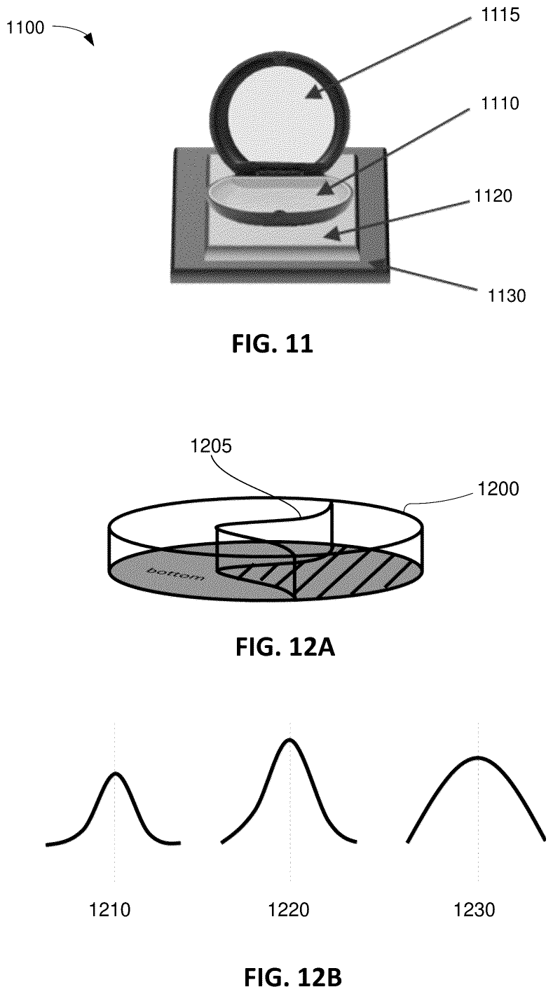

FIG. 11 shows an example NFC booster system 1100. The NFC booster system 1100 may include a case 1110, an antenna PCB 1120, and a ferrite sheet 1130. Other examples of NFC booster systems may include fewer components. For example, another NFC booster system may not include the ferrite sheet 1130. In still other examples, the antenna PCB 1120 may fit within the case 1110 instead of being disposed underneath the case 1110. The case 1110 may be an example of the case 710 of FIG. 7.

In some examples, the antenna PCB 1120 may include any feasible NFC booster including the NFC booster 610, the NFC booster 620, and/or the NFC booster 630 of FIG. 6. In some examples, the ferrite sheet 1130 may shield the antenna PCB 1120 from interference.

A shield 1115 may be attached or disposed to a lid of the case 1110. The shield 1115 may be formed from any feasible electrically conductive material, such as a conductive metal, conductive foil, or conductive coating. In some examples, the shield 1115 may be formed with a conductive paint applied to an inner surface the lid of the case 1110. The shield 1115 may increase/improve NFC performance between an NFC device within the case 1110 and a communication device (not shown). In some examples, the shield 1115 may condense an electromagnetic field associated with NFC signals, return electromagnetic fields associated with NFC signals to the NFC device and/or the antenna PCB 1120, or increase induced current in any nearby circuitry.

FIGS. 12A and 12B show an example case 1200. The case 1200 may be an example of the case 710 of FIG. 7 or the case 1110 of FIG. 11. The case 1200 may include devices or circuits to enhance or improve NFC data transfer between an NFC device held within the case 1200 and a communication device outside of the case 1200. For example, the case 1200 may include any feasible NFC booster, including any of the NFC boosters described herein.

In some examples, it may be desirable to control the position of an aligner placed in the case 1200, thereby controlling the location of an attached NFC device. Thus, the position of the NFC device may be controlled with respect to an antenna associated with any NFC booster circuit of device.

In FIG. 12A, the case 1200 may include a dental appliance guide 1205 for facilitating placement of the monitoring electronics unit of a dental appliance (e.g., an aligner, a retainer, a palatal expander) in an optimal location to enhance or improve NFC data transfer. The dental appliance guide illustrated in FIG. 12A includes a physical constraining element with protruding sidewalls that conforms to a shape of the dental appliance and thereby fixes the position and/or orientation of the dental appliance when the dental appliance is placed in line with the dental appliance guide, and therefore the attached NFC device, within the case 1200. The dental appliance guide 1205 may be added, removed, and/or modified to better conform to the aligner. Although a particular dental appliance guide is illustrated in FIG. 12A as an example, this disclosure contemplates that any suitable constraining element may be used. For example, the constraining element may be a depression in the bottom surface of the interior of the case 1200 that conforms to the shape of the dental appliance to secure the dental appliance in a fixed position and/or orientation within the case. As another example, the constraining element may be a plurality of tabs (e.g., tabs that extend vertically from the bottom surface of the interior of the case 1200) that secure the dental appliance in a fixed position and/or orientation within the case. As another example, the constraining element may be a strap or clamp that secures the dental appliance in a fixed position and/or orientation within the case.

In some examples, the dental appliance guide is releasably coupled to the case 1200, such that is removable. In such examples, the dental appliance guide 1205 may be removed and replaced with a different dental appliance guide that may be more optimal. For example, a clinician or other user may replace a current dental appliance guide 1205 with another dental appliance guide to guide an inserted dental appliance more reliably with respect to any antenna to communicate with the NFC device. Other example dental appliance guides (FIG. 12B) may include a first dental appliance guide 1210, second dental appliance guide 1220, and third dental appliance guide 1230. Although only three aligner guides are shown in this example, any number of feasible aligner guides are possible.

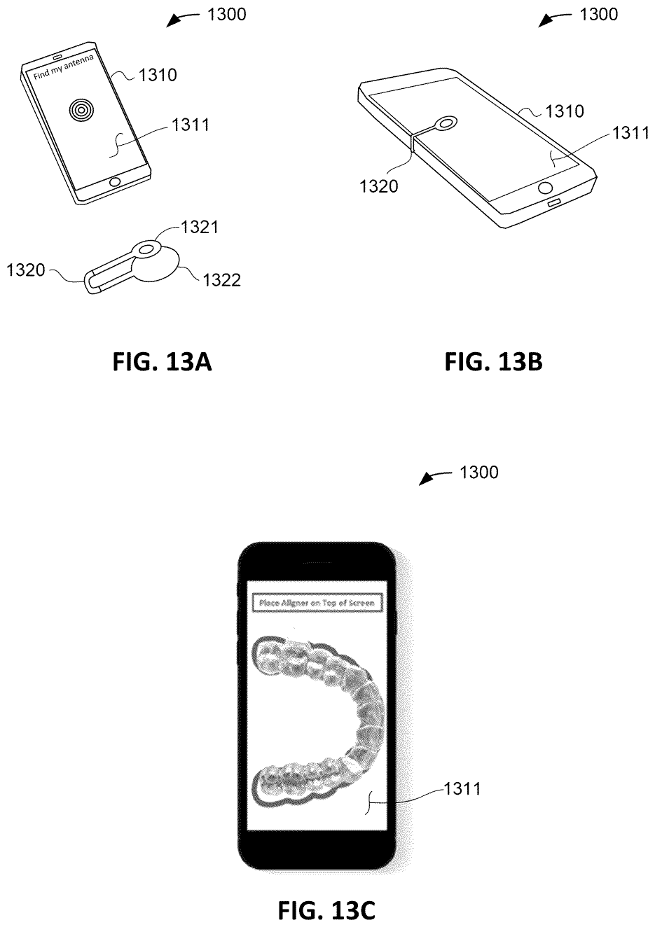

FIGS. 13A-13C show an example NFC system 1300. FIG. 13A shows some components of the NFC system 1300. The NFC system 1300 may include a communication device 1310 and an NFC booster 1320. The communication device 1310 may be any feasible device that includes at least NFC signal processing circuits. Although depicted here as a mobile phone, the communication device 1310 may be a tablet computer, laptop computer, or other feasible device. The NFC booster 1320 may be an example of the NFC boosters 610, 620, and 630 of FIG. 6.

The NFC booster 1320 may be implemented as a “clip” that attaches or slides over the communication device 1310. The NFC booster 1320 may include a first antenna 1321, a second antenna 1322, and matching circuits (not shown). The first antenna 1321 may be disposed on a first end of the clip and be positioned on a screen of the communication device 1310. The second antenna 1322 may be disposed on a second end of the clip and contact a back of the communication device 1310. The first antenna 1321 may transmit and receive NFC signals to and from an NFC device (such as an ECI device). The second antenna 1322 may transmit and receive NFC signals to and from the communication device 1310. In some examples, the communication device 1310 may guide placement of the NFC booster 1320. For example, the communication device 1310 may include a display 1311 (e.g., screen). The display 1311 may show a target or other alignment guide to position the NFC booster 1320 such that the second antenna 1322 aligns with an NFC antenna of the communication device 1310.

FIG. 13B shows another view of the NFC system 1300. The NFC booster 1320 is shown installed on (e.g., clipped around) the communication device 1310. The first antenna 1321 is on top of the display 1311. Thus, a user may place an aligner that includes ECI device over the first antenna 1321 so that the communication device 1310 can receive NFC data from the ECI device through the NFC booster 1320.

FIG. 13C shows another view of the NFC system 1300. The display 1311 may show instructions, directions, or images to guide the user's placement of the aligner and/or ECI device onto the NFC booster 1320 (not shown).

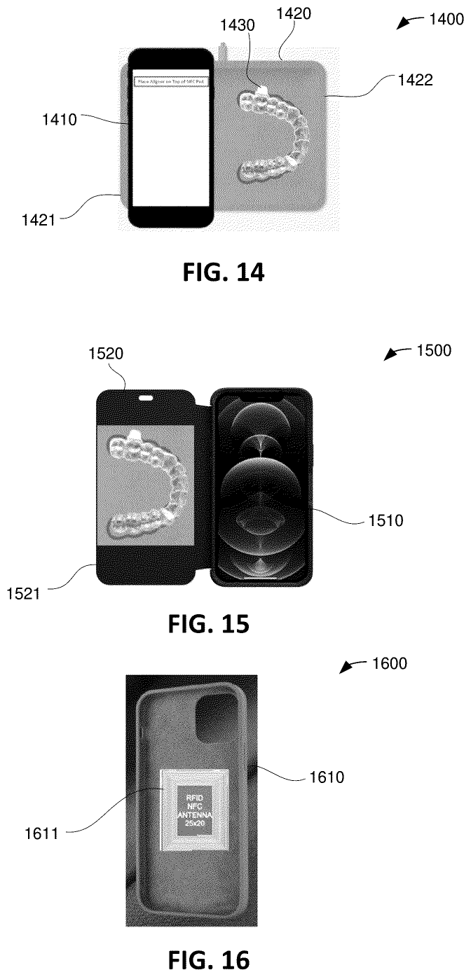

FIG. 14 shows another example NFC system 1400. The NFC system 1400 may include a communication device 1410 and a pad 1420. The communication device 1410 can be any feasible device such as a mobile phone, tablet computer, or the like. The pad 1420 may include an NFC booster (not shown), such as but not limited to the NFC boosters 610, 620, and 630 of FIG. 6.

In some examples, the pad 1420 may include a first section 1421 and a second section 1422. The first section 1421 may include a first antenna (not shown) for transmitting and receiving NFC signals to and from the communication device 1410. In some examples, the communication device 1410 may be positioned over and adjacent to the first antenna within the pad 1420. The first section 1421 may also include a non-contact (e.g., wireless) charging coil to deliver power to the communication device 1410.

The second section 1422 may include a second antenna (not shown) for transmitting and receiving NFC signals to and from an NFC device such as an ECI device 1430. In some examples, the pad 1420 may include markings or guidelines to help guide the user's placement of the NFC device on the pad 1420. In this manner, the NFC device may be placed close to the second antenna.

FIG. 15 shows another example NFC system 1500. The NFC system 1500 may include a communication device 1510 and a case 1520. The case 1520, which may be an example of a mobile phone case, may include an NFC booster (not shown), such as but not limited to the NFC boosters 610, 620, and 630 of FIG. 6. In some examples, an antenna (not shown) for transmitting and receiving NFC signals to and from the communication device 1510 may be disposed within the case 1520, such as underneath the communication device 1510. Thus, the communication device 1510 may be positioned on or near the antenna on the case 1520.

The case 1520 may include a flap 1521. The flap may include a second antenna (not shown) for transmitting and receiving NFC signals to and from an NFC device such as an ECI device. In some examples, the case 1520 may include markings or guidelines to help guide the user's placement of the NFC device on the case 1520. In this manner, the NFC device may be placed close to the second antenna.

FIG. 16 shows another example NFC system 1600. The NFC system 1600 may include a case 1610. The case 1610, which may be another example of a mobile phone case, may include an NFC booster (not shown), such as but not limited to the NFC boosters 610, 620, and 630 of FIG. 6. For example, the case 1610 may include antennas 1611 for transmitting and receiving NFC signals to and from a communication device 1510 disposed within the case 1610.

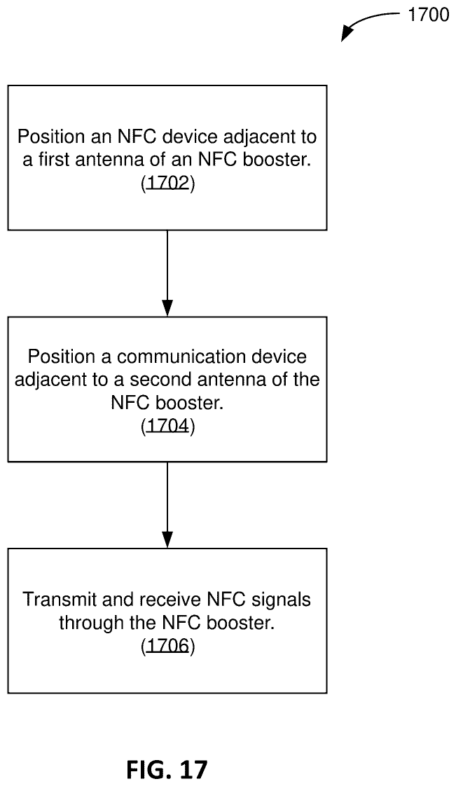

FIG. 17 is a flowchart showing an example method 1700 for providing NFC communications. Some examples may perform the operations described herein with additional operations, fewer operations, operations in a different order, operations in parallel, and some operations differently. The method 1700 is described below with respect to the NFC system 500 of FIG. 5, however, the method 1700 may be performed by or with any other suitable system or device.

The method 1700 begins in block 1702 as an NFC device is positioned near a first antenna of the NFC booster. For example, an NFC device may be positioned next to the first antenna 521 of the NFC booster 520. In some examples, the NFC booster may be included within a case, pad, clip or other feasible housing. Thus, the NFC device may be positioned within or on the case, pad, clip, or the like in order to position the NFC device near or on the first antenna. In some examples, the first antenna may be shaped or sized to enhance NFC data transfer to and from the NFC device.

Next, in block 1704, a communication device is positioned near a second antenna of the NFC booster. For example, the communication device may be positioned on or near the second antenna 522 of the NFC booster 520. The communication device may be positioned within or on the case, pad, clip, or the like in order to position the communication device near or on the second antenna,

Next, in block 1706 NFC signals are transmitted through the NFC booster. In some examples, the NFC booster 520 may include one or more matching circuits to match impedances associated with the first antenna 521 and the second antenna 522. In this manner, the one or more matching circuits may improve NFC data transfer (e.g., decrease data errors, or the like) between the NFC device and the communication device.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and examples such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for attaching a monitoring electronics unit to a dental appliance, the method comprising:
fabricating the dental appliance, wherein the dental appliance includes a mounting surface at a mounting location determined by a mounting location optimization protocol;
positioning a housing at least partially enclosing the monitoring electronics unit housing on the mounting surface; and
welding the housing to the mounting surface.

2. The method of claim 1, further comprising determining the mounting surface at the mounting location by a mounting location optimization protocol comprising: optimizing a location of the mounting surface in a digital model of the dental appliance by: iteratively adjusting one or more of a position, an angulation and an orientation of the mounting surface relative to a patient's dental arch corresponding to the digital model of the patient's dentition, until each of the position, the angulation and the orientation of the mounting surface in the digital model of the dental appliance are within a constrained range relative to the digital model of the patient's dentition and the mounting surface does not collide with the patient's teeth or with a treatment feature of the dental appliance.

3. The method of claim 1, further comprising placing the monitoring electronics unit into a housing.

4. The method of claim 1, further comprising preparing mounting surfaces of the housing.

5. The method of claim 1, wherein the dental appliance comprises an aligner.

6. The method of claim 1, wherein the monitoring electronics unit comprises an electronics compliance indicator (ECI).

7. The method of claim 1, wherein preparing the mounting surfaces include applying an infrared-absorbent spray to the mounting surfaces of the housing.

8. The method of claim 7, further comprising applying an infrared-absorbent spray to mounting surfaces of the aligner.

9. The method of claim 1, where positioning the housing includes applying clamping force to the housing.

10. The method of claim 1, wherein welding includes heating the housing and the aligner via a laser.

11. The method of claim 10, wherein the laser is a Yttrium-Aluminum Garnet laser.

12. A method for attaching a monitoring electronics unit to a dental appliance, the method comprising:
fabricating the dental appliance, wherein the dental appliance includes a mounting surface at a mounting location determined by a mounting location optimization protocol, wherein the mounting location optimization protocol: optimizing a location of the mounting surface in a digital model of the dental appliance by iteratively adjusting one or more of a position, an angulation and an orientation of the mounting surface relative to a patient's dental arch corresponding to the digital model of the patient's dentition, until each of the position, the angulation and the orientation of the mounting surface in the digital model of the dental appliance are within a constrained range relative to the digital model of the patient's dentition and the mounting surface does not collide with the patient's teeth or with a treatment feature of the aligner;
placing the monitoring electronics unit into a housing;
preparing mounting surfaces of the housing;
positioning a housing at least partially enclosing the monitoring electronics unit housing on the mounting surface; and
welding the housing to the mounting surface.

13. A method of setting a location of a mounting surface for a monitoring electronics unit on a dental appliance, the method comprising:
optimizing the location of the mounting surface having a predetermined mounting area in a digital model of the dental appliance by:
iteratively adjusting one or more of a position, an angulation and an orientation of the mounting surface relative to a patient's dental arch corresponding to a digital model of the patient's dentition corresponding to the digital model of the dental appliance, until each of the position, the angulation and the orientation of the mounting surface in the digital model of the dental appliance are each within a constraint range relative to the digital model of the patient's dentition, and the mounting surface does not collide with the patient's teeth or with a treatment feature of the aligner; and
outputting the digital model of the dental appliance, or a digital model of a mold for forming the dental appliance, including the optimized location of the mounting surface.

14. The method of claim 13, wherein iteratively adjusting the position comprises iteratively adjusting a distance from the mounting surface to a tooth buccal surface, further wherein the constraint range of the distance from the mounting surface to the tooth buccal surface is between 0.2 and 1.0 mm.

15. The method of claim 13, wherein iteratively adjusting the position comprises iteratively adjusting a distance from the mounting surface to a gingiva surface, further wherein the constraint range of the distance from the mounting surface to the gingiva surface is between 0.5 and 1.0 mm.

16. The method of claim 13, wherein iteratively adjusting the position comprises iteratively adjusting a distance from an occlusal edge of the mounting surface to a buccal cusp, further wherein the constraint range of the distance from the occlusal edge of the mounting surface to the buccal cusp is less than 1.0 mm.

17. The method of claim 13, wherein iteratively adjusting the position comprises iteratively adjusting a distance from an edge of the mounting surface to a treatment feature of the aligner, further wherein the constraint range of the distance from the occlusal edge of the mounting surface to the buccal cusp is 0.2 mm or more.

18. The method of claim 13, wherein iteratively adjusting the position comprises iteratively adjusting until the mounting surface does not collide with teeth on opposite jaw or with neighboring teeth.

19. The method of claim 13, wherein iteratively adjusting the orientation comprises iteratively adjusting an orientation between a normal of the mounting surface and a normal to a jaw arch of the patient's dentition, further wherein the constraint range of the orientation between the normal of the mounting surface and the normal to a jaw arch of the patient's dentition is between −12.5 and 12.5 degrees.

20. The method of claim 13, wherein iteratively adjusting the angulation comprises iteratively adjusting an orientation between a normal of the occlusal edge of the mounting surface and a normal to a jaw occlusal plane of the patient's dentition, further wherein the constraint range of the orientation between the normal of the occlusal edge of the mounting surface and the normal to the jaw occlusal plane is between −12.5 and 12.5 degrees.

21. The method of claim 13, further comprising manufacturing the dental appliance from the digital model of the dental appliance including the optimized location of the mounting surface.

22. A method of setting a location of a mounting surface for a monitoring electronics unit on a dental appliance, the method comprising:
receiving a digital model the dental appliance and a digital model of the patient's dentition;
optimizing the location of the mounting surface having a predetermined mounting area in the digital model of the dental appliance by:
starting from an initial location of the mounting surface, iteratively adjusting one or more of a position, an angulation and an orientation of the mounting surface relative to a patient's dental arch corresponding to the digital model of the patient's dentition, until each of the position, the angulation and the orientation of the mounting surface in the digital model of the dental appliance are within a constrained range relative to the digital model of the patient's dentition, and the mounting surface does not collide with the patient's teeth or with a treatment feature of the aligner;
outputting the digital model of the dental appliance, or a digital model of a mold for forming the dental appliance, including the optimized location of the mounting surface; and
manufacturing the dental appliance from the digital model of the dental appliance including the optimized location of the mounting surface.
